(19) United States
(12) Patent Application Publication
(10) Pub. No.: US 2025/0333495 A1
(43) Pub. Date: Oct. 30, 2025

(54) PREDICTING RESPONSE TO IL-6 ANTAGONISTS

(71) Applicant: Hoffmann-La Roche Inc., Little Falls, NJ (US)

(72) Inventors: Marina MESQUIDA, Riehen (CH); Everson NOGOCEKE, Basel (CH)

(73) Assignee: Hoffmann-La Roche Inc., Little Falls, NJ (US)

(21) Appl. No.: 19/184,178

(22) Filed: Apr. 21, 2025

Related U.S. Application Data
(63) Continuation of application No. PCT/EP2023/079391, filed on Oct. 23, 2023.

(30) Foreign Application Priority Data
Oct. 24, 2022 (EP) 22203165.0

(51) Int. Cl. C07K 16/24; A61K 39/00; A61K 39/395; A61P 27/02

(52) U.S. Cl. CPC C07K 16/248; A61K 39/3955; A61P 27/02; A61K 2039/505; A61K 2039/507; A61K 2039/54; A61K 2039/545

(57) ABSTRACT

The invention is concerned with a method of predicting response to an IL-6 angatonist such as anti-IL-6 antibody by determing the concentration of IL-6 in human aqueous humor. The invention is also concerned with an IL-6 angatonist for use in treatment of uveitis or uveitic macular edema.

### CROSS-REFERENCE TO RELATED APPLICATIONS

The present application is a continuation of the PCT Application No. PCT/EP2023/079391, filed Oct. 23, 2023, which claims priority to European Patent Application No. 22203165.0, filed Oct. 24, 2022, each of which is hereby incorporated by reference in its entirety.

### SEQUENCE LISTINGS

The instant application contains a Sequence Listing which as been submitted electronically in XML format and is hereby incorporated by reference in its entirety. Said XML copy, created on Apr. 16, 2025, is named P37806-US Sequence Listing.xml and is 10,568 bytes in size.

### FIELD OF THE INVENTION

The present invention relates to an interleukin-6 (IL-6) antagonist such as an anti-IL-6 or anti-IL-6 receptor (IL-6R) antibody for use in treatment of a patient with ophthalmic diseases with pathophysiology involving IL-6, characterized by an increased concentration of aqueous humor (AH) IL-6. The present invention also relates to a biomarker for predicting response of patients with ophthalmic diseases to an interleukin-6 (IL-6) antagonist such as an anti-IL-6 or anti-IL-6 receptor (IL-6R) monoclonal antibody. Provided herein is a method of identifying a patient with ophthalmic diseases responsive to an IL-6 or IL-6R antagonist by determining the level of IL-6 in aqueous humor (AH IL-6). The present invention further relates to an IL-6 antagonist for use in the treatment of uveitis or uveitic macular edema (UME).

### BACKGROUND OF THE INVENTION

IL-6 is among the various inflammatory mediators increased in the eye of patients with retinal diseases such as diabetic macular edema (DME), diabetic retinopathy (DR), age-related macular degeneration (AMD), retinal vein occlusion (RVO) and uveitis/uveitic macular edema (UME). IL-6 has been shown to cause loss of blood-retinal barrier function in human retinal endothelial cells in vitro, and inhibition of IL-6 trans-signaling using sgp130Fc prevents endothelial barrier disruption in retinal endothelial cells (Valle ML et al., Inhibition of interleukin-6 trans-signaling prevents inflammation and endothelial barrier disruption in retinal endothelial cells. Exp Eye Res. 2019 January). Therefore, an intraocular inhibitor of IL-6 administered locally (e.g. via intravitreal injection) can be an attractive novel therapeutic strategy to treat DME, DR, AMD, RVO or UME. However, there is currently no validated biomarker to predict response to a therapy with an anti-IL-6 antagonist.

Accordingly, there is a need for methods of predicting which patients respond particularly well to a therapy with an IL-6 antagonist. There is also a need for providing an effective treatment for uveitis and uveitic macular edema.

### SUMMARY OF THE INVENTION

Here an in vitro diagnostic immunoassay to quantify IL-6 in aqueous humor of retinal disease patients (DME, DR, AMD, RVO and UME) is developed to predict whether a patient is likely to respond to the intraocular inhibition of IL-6 by compounds delivered via intravitreal injection.

Also provided is an IL-6 antagonist for use in treatment of uveitis or uveitic macular edema.

The following numbered paragraphs define some embodiments of the present invention.
1′. An in vitro method of identifying a patient with an ophthalmic disease who is likely to respond to a therapy comprising an effective amount of an interleukin-6 (IL-6) antagonist, the method comprising determing a level of IL-6 in aqueous humor (AH IL-6) in a sample obtained from the patient, wherein an increased level of AH IL-6 relative to a reference level indicates that the patient is likely to respond to the therapy.
2′. The method of 1′, wherein the therapy is suitable for administering in the eye of the patient.
3′. The method of l′ or 2′, wherein the therapy is suitable for administering intravitreally, intraocularly, or subconjunctivally.
4′. The method of any one of 1′-3′, wherein the level of IL-6 is determined in an aqueous humor sample collected by anterior chamber paracentesis.
5′. The method of any one of 1′-4′, wherein the ophthalmic disease is selected from the group consisting of diabetic macular edema (DME), diabetic retinopathy (DR), dry eye (e.g., dry eye disease or dry eye syndrome), allergic conjunctivitis, uveitis, uveitic macular edema (UME), age-related macular degeneration (AMD) (e.g., wet AMD or dry AMD), proliferative diabetic retinopathy (PDR), rhegmatogenous retinal detachment (RRD), retinal vein occlusion (RVO), macular edema secondary to RVO, neuromyelitis optica spectrum disorder (NMOSD), myopic choroidal neovascularization, an ocular cancer, corneal transplant, corneal abrasion, or physical injury to the eye.
6′. The method of 5′, wherein the ophthalmic disease is diabetic macular edema (DME).
7′. The method of any one of 1′-6′, wherein the IL-6 antagonist is an anti-IL-6 or anti-IL-6 receptor (IL-6R) antibody or antigen binding fragment thereof.
8′. The method of 7′, wherein the IL-6 antagonist is an anti-IL-6 antibody or antigen binding fragment thereof.
9′. The method of 7′ or 8′, wherein the anti-IL-6 antibody comprises:
   i) VH CDR1 comprising the sequence of SEQ ID NO:1, VH CDR2 comprising the sequence of SEQ ID NO:2, and VH CDR3 comprising the sequence of SEQ ID NO:3; and
   ii) VL CDR1 comprising the sequence of SEQ ID NO:4, VL CDR2 comprising the sequence of SEQ ID NO:5, and VL CDR3 comprising the sequence of SEQ ID NO:6.
10′. The method of 9′, wherein the anti-IL-6 antibody comprises a heavy chain variable region comprising the sequence of SEQ ID NO:7 and a light chain variable region comprising the sequence of SEQ ID NO:8.
11′. The method of 10′, wherein the IL-6 antibody comprises a heavy chain comprising the sequence of SEQ ID NO:9 and a light chain comprising the sequence of SEQ ID NO:10.
12′. The method of any one of 1′-11′, wherein the sample is a sample obtained from the patient prior to the therapy with an IL-6 antagonist.
13′. The method of any one of claims 1′-12′, wherein the therapy further comprises an effective amount of a second therapeutic agent.
14′. The method of 13′, wherein the second therapeutic agent is a VEGF antagonist.
15′. The method of claim 14′, wherein the VEGF antagonist is an anti-VEGF antibody.
16′. A pharmaceutical composition comprising an IL-6 antagonist for use in treatment of a patient with an ophthalmic disease, wherein the patient has been determined to be likely to respond to a therapy comprising an effective amount of an IL-6 antagonist in accordance with the method of any one of 1′ to 15′.
1″. An interleukin-6 (IL-6) antagonist for use in treating a patient with an ophthalmic disease, wherein the patient has been determined to have an increased level of IL-6 in aqueous humor (AH IL-6) in a sample obtained from the patient relative to a reference level.
2″. The IL-6 antagonist for use of 1″, wherein the IL-6 antagonist is formulated as a pharmaceutical composition suitable for administering in the eye of the patient.
3″. The IL-6 antagonist for use of 2″, wherein the pharmaceutical composition is suitable for administering intravitreally, intraocularly, or subconjunctivally.
4″. The IL-6 antagonist for use of any one of 1″-3″, wherein the level of IL-6 is determined in an aqueous humor sample collected by anterior chamber paracentesis.
5″. The IL-6 antagonist for use of any one of 1″-4″, wherein the ophthalmic disease is selected from the group consisting of diabetic macular edema (DME), diabetic retinopathy (DR), dry eye (e.g., dry eye disease or dry eye syndrome), allergic conjunctivitis, uveitis, uveitic macular edema (UME), age-related macular degeneration (AMD) (e.g., wet AMD or dry AMD), proliferative diabetic retinopathy (PDR), Rhegmatogenous retinal detachment (RRD), retinal vein occlusion (RVO), macular edema secondary to RVO, neuromyelitis optica spectrum disorder (NMOSD), myopic choroidal neovascularization, an ocular cancer, corneal transplant, corneal abrasion, or physical injury to the eye.
6″. The IL-6 antagonist for use of 5″, wherein the ophthalmic disease is diabetic macular edema (DME).
7″. The IL-6 antagonist for use of any one of 1″-6″, wherein the IL-6 antagonist is an anti-IL-6 or anti-IL-6 receptor (IL-6R) antibody or antigen binding fragment thereof.
8″. The IL-6 antagonist for use of 7″, wherein the IL-6 antagonist is an anti-IL-6 antibody or antigen binding fragment thereof.
9″. The IL-6 antagonist for use of 7″ or 8″, wherein the anti-IL-6 antibody comprises:
   i) VH CDR1 comprising the sequence of SEQ ID NO:1, VH CDR2 comprising the sequence of SEQ ID NO:2, and VH CDR3 comprising the sequence of SEQ ID NO:3; and
   ii) VL CDR1 comprising the sequence of SEQ ID NO:4, VL CDR2 comprising the sequence of SEQ ID NO:5, and VL CDR3 comprising the sequence of SEQ ID NO:6.
10″. The IL-6 antagonist for use of 9″, wherein the anti-IL-6 antibody comprises a heavy chain variable region comprising the sequence of SEQ ID NO:7 and a light chain variable region comprising the sequence of SEQ ID NO:8.
11″. The IL-6 antagonist for use of 10″, wherein the IL-6 antibody comprises a heavy chain comprising the sequence of SEQ ID NO:9 and a light chain comprising the sequence of SEQ ID NO:10.
12″. The IL-6 antagonist for use of any one of claims 1″-11″, wherein the sample is a sample obtained from the patient prior to the treatment with an IL-6 antagonist.
13″. The IL-6 antagonist for use of any one of claims 1″-12″, wherein the use further comprises an effective amount of a second therapeutic agent.
14″. The IL-6 antagonist for use of 13″, wherein the second therapeutic agent is a VEGF antagonist.
15″. The IL-6 antagonist for use of 14″, wherein the VEGF antagonist is an anti-VEGF antibody.
16″. An IL-6 antagonist for use in treating a patient with uveitis or uveitic macular edema.
17″. The IL-6 antagonist for use of 16″, wherein the IL-6 antagonist is formulated as a pharmaceutical composition suitable for administering in the eye of the patient.
18″. The IL-6 antagonist for use of 17″, wherein the pharmaceutical composition is suitable for administering intravitreally, intraocularly, or subconjunctivally.
19″. The IL-6 antagonist for use of any one of claims 16″-18″, wherein the IL-6 antagonist is an anti-IL-6 or anti-IL-6 receptor (IL-6R) antibody or antigen binding fragment thereof.
20″. The IL-6 antagonist for use of claim 19″, wherein the IL-6 antagonist is an anti-IL-6 antibody or antigen binding fragment thereof.
21″. The IL-6 antagonist for use of 19″ or 20″, wherein the anti-IL-6 antibody comprises:
   i) VH CDR1 comprising the sequence of SEQ ID NO:1, VH CDR2 comprising the sequence of SEQ ID NO:2, and VH CDR3 comprising the sequence of SEQ ID NO:3; and
   ii) VL CDR1 comprising the sequence of SEQ ID NO:4, VL CDR2 comprising the sequence of SEQ ID NO:5, and VL CDR3 comprising the sequence of SEQ ID NO:6.
22″. The IL-6 antagonist for use of 21″, wherein the anti-IL-6 antibody comprises a heavy chain variable region comprising the sequence of SEQ ID NO:7 and a light chain variable region comprising the sequence of SEQ ID NO:8.
23″. The IL-6 antagonist for use of 22″, wherein the IL-6 antibody comprises a heavy chain comprising the sequence of SEQ ID NO:9 and a light chain comprising the sequence of SEQ ID NO:10.
24″. The IL-6 antagonist for use of any one of 16″-23″, wherein the IL-6 antagonist is administered intravitreally (IVT) at a dosage of 0.25 mg, 1.0 mg or 2.5 mg every 4 weeks (Q4W).

In one aspect, the present invention relates to a method of determining whether a patient with an ophthalmic disease is suitable for treatment with a therapy comprising an effective amount of an IL-6 antagonist, the method comprising determing a level of AH IL-6 in a sample obtained from the patient, wherein an increased level of AH IL-6 relative to a reference level indicates that the patient is likely to respond to the therapy.

In one aspect, the present invention relates to a method of improving the treatment effect of a therapy comprising an effective amount of an IL-6 antagonist in a patient with an ophthalmic disease, the method comprising determing a level of AH IL-6 in a sample obtained from the patient, wherein an increased level of AH IL-6 relative to a reference level indicates that the patient is likely to respond to the therapy.

In one aspect, the present invention relates to a method of treating a patient with an ophthalmic disease. The method comprises administering to a patient with an ophthalmic disease a therapy comprising an effective amount of an IL-6 antagonist, the method comprising determing a level of AH IL-6 in a sample obtained from the patient, wherein an increased level of AH IL-6 relative to a reference level indicates that the patient is likely to respond to the therapy.

In one aspect, the present invention relates to an in vitro diagnostic immunoassay comprising an anti-IL-6 antibody for use in any of the methods above.

In one aspect, the present invention relates to the use of an IL-6 antagonist for the manufacture of a medicament for the treatment of a patient with an ophthalmic disease, wherein the patient has been determined to be likely to respond to a therapy comprising an effective amount of an IL-6 antagonist in accordance with any of the methods above.

These and other embodiments are further described in the detailed description below.

### BRIEF DESCRIPTION OF THE FIGURES

FIG. 1: Absolute BCVA over time in UME patients participating in the Part 4 of the DOVETAIL study. Data is shown as mean BCVA per dose group (2.5 mg, 1 mg or 0.5 mg RO7200220). RO7200220 was administered intravitreal (IVT) 3 times, 4 weeks apart (Day 1, Day 28 and Day 56).

FIG. 2: BCVA change from baseline (Day 1) over time in UME patients participating in the Part 4 of the DOVETAIL study. Data is shown as mean change from baseline per dose group (2.5 mg, 1 mg or 0.5 mg RO7200220). RO7200220 was administered IVT 3 times, 4 weeks apart (Day 1, Day 28 and Day 56).

FIG. 3: Absolute CST over time in UME patients participating in the Part 4 of the DOVETAIL study. Data is shown as mean CST per dose group (2.5 mg, 1 mg or 0.5 mg RO7200220). RO7200220 was administered IVT 3 times, 4 weeks apart (Day 1, Day 28 and Day 56).

FIG. 4: CST change from baseline (Day 1) over time UME patients participating in the Part 4 of the DOVETAIL study. Data is shown as mean change from baseline per dose group (2.5 mg, 1 mg or 0.5 mg RO7200220). RO7200220 was administered IVT 3 times, 4 weeks apart (Day 1, Day 28 and Day 56).

FIG. 5: Absolute subretinal fluid (SRF) volume over time in UME patients participating in the Part 4 of the DOVETAIL study. Data is shown as mean and standard error of the UME population (n=23). RO7200220 was administered IVT 3 times, 4 weeks apart (Day 1, Day 28 and Day 56), as indicated by the grey dotted lines. SRF volumes were measured on SD-OCT images in the 3.0 mm radius ring of the Early Treatment Diabetic Retinopathy Study (ETDRS) grid.

FIG. 6: Absolute intraretinal fluid (IRF) volume over time in UME patients participating in the Part 4 of the DOVETAIL study. Data is shown as mean and standard error of the UME population (n=23). RO7200220 was administered IVT 3 times, 4 weeks apart (Day 1, Day 28 and Day 56), as indicated by the grey dotted lines. IRF volumes were measured on SD-OCT images in the 3.0 mm radius ring of the Early Treatment Diabetic Retinopathy Study (ETDRS) grid.

FIG. 7: Baseline AH IL-6 vs Change from baseline in CST of DME patients participating in the Part 1 of the DOVETAIL study. Change in CST as average CST at 70 and 98 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration.

FIG. 8: Baseline AH IL-6 vs Change from baseline in BCVA of DME patients participating in Part 1 of the DOVETAIL study. Change in BCVA as average BCVA at 70 and 98 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration.

FIG. 9: Baseline AH IL-6 vs Change from baseline in CST of UME patients participating in the Part 4 of the DOVETAIL study. Change in CST as average CST at 56 and 84 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration.

FIG. 10: Baseline AH IL-6 vs Change from baseline in BCVA of UME patients participating in Part 4 of the DOVETAIL study. Change in BCVA as average BCVA at 56 and 84 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration.

FIG. 11: Absolute BCVA over time in UME patients participating in the Part 4 of the DOVETAIL study. Data is shown as mean BCVA per dose group (2.5 mg, 1 mg or 0.5 mg RO7200220). RO7200220 was administered intravitreal (IVT) 3 times, 4 weeks apart (Day 1, Day 28 and Day 56). Error bars represent standard error. Baseline is the patient's last observation prior to initiation of study drug. One patient in the 1 mg arm had their Day 28/56 doses delayed. Subsequently, this patient received a dose at the Day 42 and Day 84 Visits. Windowing was applied to this patient and the visits on days 42, 59, 81, 123, 150 and 207 were mapped to the Day 42, Day 56, Day 84, Day 112, Day 140 and Day 196 visits, respectively.

FIG. 12: BCVA change from baseline (Day 1) over time in UME patients participating in the Part 4 of the DOVETAIL study. Data is shown as mean change from baseline per dose group (2.5 mg, 1 mg or 0.5 mg RO7200220). RO7200220 was administered IVT 3 times, 4weeks apart (Day 1, Day 28 and Day 56). Error bars represent standard error. Baseline is the patient's last observation prior to initiation of study drug. One patient in the 1 mg arm had their Day 28/56 doses delayed. Subsequently, this patient received a dose at the Day 42 and Day 84 Visits. Windowing was applied to this patient and the visits on days 42, 59, 81, 123, 150 and 207 were mapped to the Day 42, Day 56, Day 84, Day 112, Day 140 and Day 196 visits, respectively.

FIG. 13: Absolute CST over time in UME patients participating in the Part 4 of the DOVETAIL study. Data is shown as mean CST per dose group (2.5 mg, 1 mg or 0.5 mg RO7200220). RO7200220 was administered IVT 3 times, 4 weeks apart (Day 1, Day 28 and Day 56). Error bars represent standard error. Baseline is the patient's last observation prior to initiation of study drug. One patient in the 1 mg arm had their Day 28/56 doses delayed. Subsequently, this patient received a dose at the Day 42 and Day 84 Visits. Windowing was applied to this patient and the visits on days 42, 59, 81, 123, 150 and 207 were mapped to the Day 42, Day 56, Day 84, Day 112, Day 140 and Day 196 visits, respectively.

FIG. 14: CST change from baseline (Day 1) over time UME patients participating in the Part 4 of the DOVETAIL study. Data is shown as mean change from baseline per dose group (2.5 mg, 1 mg or 0.5 mg RO7200220). RO7200220 was administered IVT 3 times, 4 weeks apart (Day 1, Day 28 and Day 56). Error bars represent standard error. Baseline is the patient's last observation prior to initiation of study drug. One patient in the 1 mg arm had their Day 28/56 doses delayed. Subsequently, this patient received a dose at the Day 42 and Day 84 Visits. Windowing was applied to this patient and the visits on days 42, 59, 81, 123, 150 and 207 were mapped to the Day 42, Day 56, Day 84, Day 112, Day 140 and Day 196 visits, respectively.

FIG. 15: Absolute subretinal fluid (SRF) volume over time in UME patients participating in the Part 4 of the DOVETAIL study. Data is shown as mean and standard error of the UME population (n=37). RO7200220 was administered IVT 3 times, 4 weeks apart (Day 1, Day 28 and Day 56), as indicated by the grey dotted lines. SRF volumes were measured on SD-OCT images in the 3.0 mm radius ring of the Early Treatment Diabetic Retinopathy Study (ETDRS) grid.

FIG. 16: Absolute intraretinal fluid (IRF) volume over time in UME patients participating in the Part 4 of the DOVETAIL study. Data is shown as mean and standard error of the UME population (n=37). RO7200220 was administered IVT 3 times, 4 weeks apart (Day 1, Day 28 and Day 56), as indicated by the grey dotted lines. IRF volumes were measured on SD-OCT images in the 3.0 mm radius ring of the Early Treatment Diabetic Retinopathy Study (ETDRS) grid.

FIG. 17: Baseline AH IL-6 vs Change from baseline in CST of DME patients participating in the Part 1 of the DOVETAIL study. Change in CST as average CST at 70 and 98 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration. One patient in the 1 mg arm had their Day 28/56 dose delayed (dosed at Day 42 and Day 84). Visits conducted on study days 81 and 101 for this patient were mapped to Day 84 and Day 112 Visits.

FIG. 18: Baseline AH IL-6 vs Change from baseline in BCVA of DME patients participating in Part 1 of the DOVETAIL study. Change in BCVA as average BCVA at 70 and 98 days relative to the baseline (i.e. Day 1, the day of the first RO7200220 injection), assuming at least one of these measurements is available. On x-axis is log base 10 of baseline (Day 1, prior to RO7200220 injection) AH IL-6 concentration. One patient in the 1 mg arm had their Day 28/56 dose delayed (dosed at Day 42 and Day 84). Visits conducted on study days 81 and 101 for this patient were mapped to Day 84 and Day 112 Visits.

### DETAILED DESCRIPTION OF THE EMBODIMENTS

Provided here are interleukin-6 (IL-6) antagonists such as an anti-IL-6 or anti-IL-6 receptor (IL-6R) antibodies for use in treatment of a patient with ophthalmic diseases with pathophysiology involving IL-6, characterized by an increased level of aqueous humor (AH) IL-6. Also provided here are methods of identifying patients with ophthalmic diseases who are responsive to a therapy comprising an effective amount of an IL-6 antagonist by determining IL-6 levels in AH.

Although molecular mediators of BRB breakdown exert their activity within the retina, clinical investigation of the relevance of these molecules in retinal diseases such as UME and DME patients has relied on analysis of surrogate specimens such as vitreous and aqueous humor. Retina samples of patients cannot be collected due to the invasive nature of the sampling procedure and its deleterious consequences to the patients. Vitreous is in close contact with the retina and its molecular composition is believed to contain factors released by the retinal cells.

Nonetheless, vitreous sample collection is typically only possible upon eligible vitrectomy, limiting its use for analytical purposes. Aqueous humor is the fluid that fills the anterior chamber of the eye and is more easily collected than vitreous.

### Definitions

### IL-6 Antagonist

The term “IL-6 antagonist (IL-6a)” refers to a molecule that can bind to IL-6 or IL-6R, and inhibits or reduces at least one IL-6 activity. IL-6 activity can include one or more of the following: binding to gp130; activation of the IL-6 signaling pathway; activation of a JAK kinase, e.g., phosphorylation of a target of a JAK kinase; activation of a STAT protein, e.g., phosphorylation of a STAT protein; and/or expression of a STAT-target gene.

In one aspect, an IL-6a described herein specifically binds to site II (site 2) of an IL-6 and is useful for treatment of IL-6 related diseases, e.g., IL-6 related eye diseases and certain other diseases as described herein.

In one aspect, the IL-6a features one or more of the following properties: has high affinity for either free IL-6 (e.g., soluble IL-6) or bound IL-6 (e.g., IL-6 bound to an IL-6 receptor) or both free and bound IL-6; is relatively stable in an organism; can inhibit binding to gp130 of an IL-6 bound to an IL-6R (termed herein an IL-6/IL-6R complex or IL-6/IL-6R); and/or can have a therapeutic effect.

In one aspect, the IL-6a is an antibody or is a fragment derived from an antibody. For example, an IL-6a is a high affinity, humanized Fab that can specifically bind to site II of an IL-6 and potently blocks both cis-and trans-IL-6 signaling. In another example, the IL-6a is a full length antibody, e.g., an IgG1 or IgG2 antibody.

In one aspect, the IL-6a selectively binds to site II of IL-6 and provides broad inhibition of IL-6 signaling because such molecules can inhibit the binding of gp130 to IL-6, regardless of whether the IL-6 is free or bound to membrane IL-6R or sIL-6R. Furthermore, targeting the ligand (IL-6) as opposed to the IL-6 receptor can avoid receptor mediated clearance and toxicity due to ADCC (antibody-dependent cell-mediated cytotoxicity).

Because IL-6 plays both pathologic and protective roles in disease, use of an IL-6a to treat a disease associated with increased IL-6 can improve certain aspects of a condition, but may also cause significant adverse effects, e.g., systemic effects. This duality of IL-6 pathways (i.e., the ability to have desirable and/or undesirable effects) can make it undesirable to treat an IL-6 associated disorder with a systemic inhibitor. Accordingly, the compositions and methods provided herein can be useful for treatments that inhibit at least one IL-6 activity, but do not have an undue effect on positive activities of IL-6, in part because the compositions can be formulated for local delivery, e.g., for local delivery to the eye. For example, in one aspect, the IL-6a is designed to be of a size suitable for delivery to a particular site. In some embodiments, the IL-6a is a full-length antibody. In one aspect, the IL-6a is derived from an antibody and is in a format that may have longer residency in a particular compartment of the eye, e.g., the vitreous of the eye, and limited systemic leakage. In one aspect, the IL-6a is a modified antibody (e.g., an antibody with a modified Fc domain) that has longer residency in the vitreous of the eye and/or more limited systemic leakage compared with a corresponding unmodified antibody. In some embodiments, the IL-6a is an IgG2 antibody.

In one aspect, the IL-6a is a relatively small IL-6a such as a fragment of an IL-6 antibody or other derivative of an antibody that is less than a full length antibody, e.g., a Fab that is derived from an IL-6 antibody. In one aspect, an IL-6a is in a format that can pass from one part of a tissue to another with increased kinetics compared to a corresponding full-length IL-6 antibody. In some embodiments, the IL-6a is a Fab that has been engineered to be a larger molecule, which is more likely to have increased residence in the location to which it was delivered compared to the Fab alone, e.g., the IL-6a is dimerized through Fc domain. In one aspect, the Fc domain has been engineered such that the Fc moiety has ablated or reduced FcRn binding that can reduce systemic accumulation compared to the same IL-6 binding entity that includes a wild-type Fc. The engineered Fc domain can be, e.g., an IgG1 domain or an IgG2 domain.

Typically, the IL-6 antagonists described herein have a sufficiently high affinity for their target, IL-6 or IL-6R, to be effective in ameliorating at least one undesirable effect of IL-6 and are sufficiently stable to be useful as therapeutics.

In general, the PK of an IL-6a suitable for use in the eye has a sufficiently long half life in the site of delivery, e.g., the vitreous, to provide a therapeutic effect. For example, the PK can be a half-life of at least 8 days, 10 days, 14 days, 21 days, 28 days, or 30 days.

### Identification of IL-6 Antagonists Binding to Site II

In general, any method known in the art can be used to generate a molecule that can bind to an IL-6, for example, polypeptide libraries or molecular libraries can be screened for candidate compounds in an assay for the ability of a polypeptide or compound to bind to IL-6. Once such a candidate compound is identified, the binding site of the compound can be determined using methods known in the art. For example, a molecule can be tested for the ability to bind to wild type IL-6 and the binding compared to the ability of the compound to bind to an IL-6 mutated in site I, site II, or site III. In one aspect, an IL-6a as described herein retains the ability to bind to an IL-6/IL-6Rα complex and to IL-6, and prevents binding of IL-6/IL-6Rα to gp130. In one aspect, an IL-6a as described herein can compete with gp130 for binding to IL-6/IL-6Rα complex, e.g., by binding to site II of IL-6. Such binding activities can be assayed using methods known in the art.

IL-6a candidates can be tested, for example, using an HEK-Blue™ IL-6 assay system (InvivoGen, San Diego). HEK-Blue™ IL-6 cells are HEK293 cells that are stably transfected with human IL-6R and a STAT3-inducible SEAP reporter gene. In the presence of IL-6, STAT3 is activated and SEAP is secreted. SEAP is assessed using, for example, QUANTI-Blue™ (InvivoGen, San Diego). Addition of an IL-6a to the cells prevents secretion or decreases the level of SEAP as a result of inhibiting both free and soluble receptor bound IL-6.

K_{D} refers to the binding affinity equilibrium constant of a particular antibody-antigen interaction or antibody fragment-antigen interaction. In one aspect, an antibody or antigen binding fragment described herein binds to IL-6 or IL-6R with a K_{D} that is less than or equal to 250 pM, e.g., less than or equal to 225 pM, 220 pM, 210 pM, 205 pM,150 pM, 100 pM, 50 pM, 20 pM, 10 pM, or 1 pM. K_{D} can be determined using methods known in the art, for example using surface plasmon resonance, for example, using the BiaCore™ system.

K_{off} refers to the dissociation rate constant of a particular antibody-antigen interaction or antibody fragment-antigen complex. The dissociation rate constant can be determined using surface plasmon resonance, for example using the BiaCore™ system. A relatively slow K_{off} can contribute to desirable features of a therapeutic, e.g., permitting less frequent administration of the inhibitor to a subject in need of such treatment.

### Specificity

In one aspect, an IL-6a described herein binds specifically to a target, e.g., an IL-6. In general, “specific binding” as used herein indicates that a molecule preferentially binds to a selected molecule and displays much lower binding affinity for one or more other molecules. In embodiments, the binding affinity for another molecule is 1, 2, 3 or more orders of magnitude lower than the binding affinity for the target.

As discussed supra, IL-6 can be present as free IL-6 and as IL-6 bound to soluble IL-6Rα. Site II of IL-6 is an optimal target for an IL-6 antagonist compared to an inhibitor that that binds to site I of an IL-6. A site I inhibitor may inhibit binding of free IL-6 to IL-6Rα. However, such an inhibitor cannot prevent activity initiated by pre-existing IL-6/IL-6R complexes except by replacement limited by the k_{off} of the complex. Another alternative, an inhibitor that binds to an IL-6Rα, is less suitable because it may have limited ability to prevent IL-6 activity unless it is present in saturating concentrations. Because the amount of IL-6 receptor is generally quite high compared to the amount of IL-6, this approach may require the administration of an undesirably large amount of a composition that inhibits IL-6 activity by binding to the receptor. In one aspect, the IL-6a described herein can block the activity of IL-6 even when IL-6 is bound to IL-6R. Accordingly, an advantage of an IL-6a as described herein is that relatively less of the composition may need to be administered to achieve a therapeutic effect compared to an inhibitor targeting an IL-6 receptor. Anti-receptor antibodies have been reported to be cleared rapidly by receptor mediated clearance significantly limiting their PK, therefore requiring larger doses, more frequent dosing, or both. Additionally, both anti-receptor and anti-site I IL-6 antibodies pose a problem in that they significantly increase the tissue concentration of IL-6 by disrupting the normal receptor mediated clearance pathway of the ligand, thereby exposing the subject to potentially undesirable levels of IL-6 in a tissue. Furthermore, use of an inhibitor targeting IL-6Rα may necessitate the presence of the inhibitor near both sites at which inhibition is sought and a site at which it is not desirable, e.g., systemic treatment. Use of an IL-6a that binds site II, the site to which gp130 binds, permits inhibition via free IL-6 as well as IL-6 that is bound to an IL-6R, but has not yet activated an IL-6 pathway via gp130. Accordingly, without wishing to be bound by theory, the IL-6 antagonists described herein are designed to bind to both forms of IL-6 (soluble and receptor bound), specifically the IL-6 antagonists bind to site II of IL-6, which is accessible in both forms. Compositions containing an IL-6a as described herein can inhibit both cis and trans signaling by IL-6.

In one aspect, compositions and methods provided herein are designed to provide an effective IL-6 blockade sufficient to treat at least one sign or symptom of an IL-6 associated disorder, for example, inhibiting angiogenesis and/or inflammation.

Compositions described herein are useful for treating eye diseases characterized by an undesirably high level of IL-6, e.g., in the vitreous (see Yuuki et al., J Diabetes Compl 15:257 (2001); Funatsu et al., Ophthalmology 110:1690, (2003); Oh et al., Curr Eye Res 35:1116 (2010); Noma et al., Eye 22:42 (2008); Kawashima et al., Jpn J Ophthalmol 51:100 (2007); Kauffman et al., Invest Ophthalmol Vis Sci 35:900 (1994); Miao et al., Molec Vis 18:574(2012)).

In general, an IL-6a as described herein is a potent antagonist of IL-6 signaling. In one aspect, an IL-6a described herein has a high affinity for IL-6, for example, an IC50 less than or equal to 100 pM in an HEK-Blue IL-6 assay using 10 pM IL-6. High affinity of an IL-6a can be determined based on the K_{D} of the IL-6a, for example, a K_{D} of less than or equal to 1 nM, less than or equal to 500 pM, less than or equal to 400 pM, less than or equal to 300 pM, less than or equal to 240 pM, or less than or equal to 200 pM.

To produce a biologic IL-6a (e.g., a protein or polypeptide such as an antibody, fragment, or derivative thereof) that is useful for treating a disorder associated with increased IL-6 expression or activity, typically it is desirable that the biologic IL-6a have high productivity. For example, a suitable productivity is greater than or equal to 1 g/L (e.g., greater than or equal to 2 g/L, greater than or equal to 5 g/L, or greater than or equal to 10 g/L).

To effectively administer an IL-6 antagonist, it is necessary that the inhibitor have solubility compatible with the concentration at which it will be administered. For example, in the case of a full-length antibody IL-6a, the solubility is greater than or equal to 20 mg/ml, greater than or equal to 10 mg/ml, greater than or equal to 5 mg/ml, or greater than or equal to 1 mg/ml.

Furthermore, to be a viable treatment, the inhibitor must have high stability at the body temperature of the delivery and activity sites as well as storage stability. In one aspect, the inhibitor has a Tₘ of greater than or equal to 60° C. (e.g., greater than or equal to 60° C., greater than or equal to 62.5° C., greater than or equal to 65° C., greater than or equal to 70° C., greater than or equal to 73° C., or greater than or equal to 75° C.). In one aspect, the inhibitor has a Tₒₙₛₑₜ of greater than or equal to 45° C., e.g., greater than or equal to 50° C., greater than or equal to 51° C., greater than or equal to 55° C., or greater than or equal to 60° C. Methods of determining the Tₘ and Tₒₙₛₑₜ can be determined using methods known in the art.

Antagonists having the desired features can be selected from suitable types of molecules known in the art, for example antibodies, including fragments and derivatives of an IL-6 site II targeted antibody that generally retains or maintains sufficient features of the parent IL-6 antibody (e.g., desired binding properties). Such antagonists include F_{ab} fragments, scFvs, F_{ab} fragments engineered to include an Fc moiety, and full-length antibodies engineered to have a framework different from the parent IL-6 site II targeted antibody.

In one aspect, the IL-6a disclosed herein comprises a human antibody antigen-binding site that can compete or cross-compete with an antibody or fragment thereof that can bind to site II of IL-6. For example, the antibody or fragment thereof can be composed of a VH domain and a VL domain disclosed herein, and the VH and VL domains comprise a set of CDRs of an IL-6/site II binding antibody disclosed herein.

Any suitable method may be used to determine the domain and/or epitope bound by an IL-6a, for example, by mutating various sites on an IL-6. Those sites in which mutations prevent or decrease binding of the IL-6a and the IL-6 ligand are involved either directly in binding to the IL-6a or indirectly affect the binding site, e.g., by affecting conformation of the IL-6. Other methods can be used to determine the amino acids bound by an IL-6a. For example, a peptide-binding scan can be used, such as a PEPSCAN-based enzyme linked immuno assay (ELISA). In a peptide-binding scan of this type, short overlapping peptides derived from the antigen are systematically screened for binding to a binding member. The peptides can be covalently coupled to a support surface to form an array of peptides. Peptides can be in a linear or constrained conformation. A constrained conformation can be produced using peptides having a terminal cysteine (cys) residue at each end of the peptide sequence. The cys residues can be covalently coupled directly or indirectly to a support surface such that the peptide is held in a looped conformation. Accordingly, a peptide used in the method may have a cys residue added to each end of a peptide sequence corresponding to a fragment of the antigen. Double looped peptides can also be used, in which a cys residue is additionally located at or near the middle of the peptide sequence. The cys residues can be covalently coupled directly or indirectly to a support surface such that the peptides form a double-looped conformation, with one loop on each side of the central cys residue. Peptides can be synthetically generated, and cys residues can therefore be engineered at desired locations, despite not occurring naturally in the IL-6 site II sequence. Optionally, linear and constrained peptides can both be screened in a peptide-binding assay. A peptide-binding scan may involve identifying (e.g., using an ELISA) a set of peptides to which the binding member binds, wherein the peptides have amino acid sequences corresponding to fragments of an IL-6a (e.g., peptides that include about 5, 10, or 15 contiguous residues of an IL-6a), and aligning the peptides in order to determine a footprint of residues bound by the binding member, where the footprint comprises residues common to overlapping peptides. Alternatively or additionally the peptide-binding scan method can be used to identify peptides to which the IL-6a binds with at least a selected signal: noise ratio.

Other methods known in the art can be used to determine the residues bound by an antibody, and/or to confirm peptide-binding scan results, including for example, site directed mutagenesis (e.g., as described herein), hydrogen deuterium exchange, mass spectrometry, NMR, and X-ray crystallography.

Typically, an IL-6a useful as described herein is a human antibody molecule, a humanized antibody molecule, or binding fragment thereof. In general, the antibody is a monoclonal antibody. The origin of such an antibody can be human, murine, rat, camelid, rabbit, ovine, porcine, or bovine and can be generated according to methods known to those in the art.

The term “antibody molecule,” as used herein, refers to a protein, or polypeptide sequence derived from an immunoglobulin molecule which specifically binds with an antigen. The antibody molecule can be a full-length antibody or a fragment thereof, e.g., an antigen binding fragment thereof. Antibodies can be polyclonal or monoclonal, multiple or single chain, or intact immunoglobulins, and may be derived from natural sources or from recombinant sources. Antibodies can be tetramers of immunoglobulin molecules. Antibody fragments or antigen binding fragments refer to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or

VH), camelid VHH domains, and multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Patent No.: 6,703,199, which describes fibronectin polypeptide minibodies).

### Exemplary IL-6 Antibodies

In general, an IL-6a comprises at least the CDRs of an antibody that can specifically bind to an IL-6 (e.g., a human IL-6), e.g., to site II of an IL-6. The structure for carrying a CDR or a set of CDRs of the invention can be an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains can be determined by reference to Kabat, et al., 1983 (National Institutes of Health), and updates thereof findable under “Kabat” using any internet search engine.

An IL-6a, as disclosed herein, is typically an antibody molecule that generally comprises an antibody VH domain and/or VL domain. A VH domain comprises a set of heavy chain CDRs (VH CDRs), and a VL domain comprises a set of light chain CDRs (VLCDRs). Examples of such CDRS are provided herein in the Examples. An antibody molecule can comprise an antibody VH domain comprising a VH CDR1, VH CDR2 and VH CDR3 and a framework. It can also comprise an antibody VL domain comprising a VL CDR1, VL CDR2 and VL CDR3 and a framework.

Disclosed herein are IL-6 antagonists comprising a VH CDR1 and VH CDR2 and VH CDR3 such as those disclosed herein and a VL CDR1 and VL CDR2 and VL CDR3 such as those disclosed herein. The CDRs can be derived from one or more antibodies. For example, the VL CDRs can be derived from the same or a different antibody as the VH CDRs.

In one aspect, the anti-IL-6 antibody or antigen binding fragment thereof, comprises a VH CDR1 comprising the sequence of SEQ ID NO:1, a VH CDR2 comprising the sequence of SEQ ID NO:2, and a VH CDR3 comprising the sequence of SEQ ID NO:3.

In one aspect, the anti-IL-6 antibody or antigen binding fragment thereof, comprises a VL CDR1 comprising the sequence of SEQ ID NO:4, a VL CDR2 comprising the sequence of SEQ ID NO:5, and a VL CDR3 comprising the sequence of SEQ ID NO:6.

In one aspect, the anti-IL-6 antibody or antigen binding fragment thereof, comprises a VH CDR1 comprising the sequence of SEQ ID NO:1, a VH CDR2 comprising the sequence of SEQ ID NO:2, a VH CDR3 comprising the sequence of SEQ ID NO:3, a VL CDR1 comprising the sequence of SEQ ID NO:4, a VL CDR2 comprising the sequence of SEQ ID NO: 5, and a VL CDR3 comprising the sequence of SEQ ID NO:6.

In one aspect, the anti-IL-6 antibody or antigen binding fragment thereof, comprises a heavy chain variable region comprising a sequence that is at least 90, 91, 92, 93, 94, 95, 96, 97, 98, or 99% identical with SEQ ID NO:7.

In one aspect, the anti-IL-6 antibody or antigen binding fragment thereof, comprises a light chain variable region comprising a sequence that is at least 90, 91, 92, 93, 94, 95, 96, 97, 98, or 99% identical with SEQ ID NO:8.

In one aspect, the anti-IL-6 antibody or antigen binding fragment thereof, comprises a heavy chain variable region comprising the sequence of SEQ ID NO:7 and a light chain variable region comprising the sequence of SEQ ID NO:8.

In one aspect, the anti-IL-6 antibody or antigen binding fragment thereof, comprises a heavy chain comprising a sequence that is at least 90, 91, 92, 93, 94, 95, 96, 97, 98, or 99% identical with SEQ ID NO:9.

In one aspect, the anti-IL-6 antibody or antigen binding fragment thereof, comprises a light chain comprising a sequence that is at least 90, 91, 92, 93, 94, 95, 96, 97, 98, or 99% identical with SEQ ID NO:10.

In one aspect, the anti-IL-6 antibody or antigen binding fragment thereof, comprises a heavy chain comprising the sequence of SEQ ID NO:9 and a light chain comprising the sequence of SEQ ID NO:10.

In one aspect, the anti-IL-6 antibody is RO7200220, which comprises a VH CDR1 comprising the sequence of SEQ ID NO:1, a VH CDR2 comprising the sequence of SEQ ID NO: 2, a VH CDR3 comprising the sequence of SEQ ID NO:3, a VL CDR1 comprising the sequence of SEQ ID NO:4, a VL CDR2 comprising the sequence of SEQ ID NO:5, and a VL CDR3 comprising the sequence of SEQ ID NO:6; a heavy chain variable region comprising the sequence of SEQ ID NO:7 and a light chain variable region comprising the sequence of SEQ ID NO:8; or a heavy chain comprising the sequence of SEQ ID NO:9 and a light chain comprising the sequence of SEQ ID NO:10.

In embodiments, the antibody molecule, e.g., antibody or antigen binding fragment, has increased affinity for human IL-6 and/or increased potency compared with an antibody or antigen binding fragment comprising one or more corresponding sequences of EBI-029, or sequences of an antibody described in WO2014/074905, hereby incorporated by reference in its entirety. In one aspect, antibody or antigen binding fragment has increased affinity for human IL-6 and/or increased potency compared with tocilizumab.

An IL-6a as described herein can comprise antibody constant regions or parts thereof, e.g., human antibody constant regions or parts thereof. For example, a VL domain may be attached at its C-terminal end to antibody light chain constant domains including human CK or CL chains. Similarly, an IL-6a based on a VH domain can be attached at its C-terminal end to all or part (e.g., a CH1 domain) of an immunoglobulin heavy chain derived from any antibody isotype, e.g. IgG, IgA, IgE and IgM and any of the isotype sub-classes, particularly IgG1, IgG2, IgG3 and IgG4. In embodiments, the antibody or antigen binding fragment is engineered to reduce or eliminate ADCC activity.

In one aspect, the antibody of the invention is an IgG2 antibody. In one aspect, the antibody of the invention comprises an IgG2 framework, IgG2 constant region, or IgG2 Fc region.

IgG2 antibodies can exist as three major structural isoforms: IgG2-A, IgG2-B, and IgG2-A/B (Wypych J. et al. Journal of Biological Chemistry. 2008, 283:16194-16205). This structural heterogeneity is due to different configurations of the disulfide bonds that link the Fab arms to the heavy chain hinge region. In the IgG2-A isoform, there are no disulfide bonds linking the Fab arms to the hinge region. In the IgG2-B isoform, both Fab arms have disulfide bonds linking the heavy and light chain to the hinge region. The IgG2-A/B isoform is a hybrid between the IgG2-A and IgG2-B isoforms, with only one Fab arm having disulfide bonds linking the heavy and light chain of the one Fab arm to the hinge region. The conversion of an IgG2 antibody between two or all of the different structural isoforms, also referred to as disulfide shuffling, occurs naturally in vivo and in vitro for both naturally-occurring and recombinant antibodies. As a result, formulations of IgG2 antibodies in the art comprise a heterogeneous mixture of IgG2-A, IgG2-B, and IgG2-A/B isoforms. The different IgG2 isoforms can have unique and different functional properties, such as differences in stability, aggregation, viscosity, Fc receptor binding, or potency. Presence of multiple isoforms or increased levels of a particular isoform in a IgG2 antibody formulation can negatively affect stability, aggregation, or potency. Some fragments of an IgG2 antibody that can still undergo disulfide shuffling and exist in any of the structural isoforms A, A/B, and/or B can be readily envisioned, e.g., fragments that retain the residues that participate in the shuffling disulfide bonds, e.g., the fragment comprises at least an IgG2 hinge region.

Antibody fragments that comprise an antibody antigen-binding site include, but are not limited to, molecules such as Fab, Fab′, Fab′-SH, scFv, Fv, dAb, Fd, and disulfide stabilized variable region (dsFv). Various other antibody molecules including one or more antibody antigen-binding sites can be engineered, including for example F(ab′)2, F(ab)3, diabodies, triabodies, tetrabodies, and minibodies. Examples of antibody molecules and methods for their construction and use are described in Holliger and Hudson, 2005, Nat Biotechnol 23:1126-1136. Non-limiting examples of binding fragments are a Fab fragment composed of VL, VH, constant light chain domain (CL) and constant heavy chain domain 1 (CH1) domains; an Fd fragment composed of VH and CH1 domains; an Fv fragment composed of the VL and VH domains of a single antibody; a dAb fragment composed of a VH or a VL domain; isolated CDR regions; an F(ab′)2 fragment, a bivalent fragment comprising two linked Fab fragments; a single chain Fv molecule (scFv), in which a VH domain and a VL domain are linked by a peptide linker which allows the two domains to associate to form an antigen binding site; a bispecific single chain Fv dimer (for example as disclosed in WO 1993/011161) and a diabody, which is a multivalent or multispecific fragment constructed using gene fusion (for example as disclosed in WO94/13804). Fv, scFv, or diabody molecules can be stabilized by the incorporation of disulfide bridges linking the VH and VL domains. Minibodies comprising an scFv joined to a CH3 domain can also be used as an IL-6a. Other fragments and derivatives of an antibody that can be used as an IL-6a include a Fab′, which differs from a Fab fragment by the addition of a few amino acid residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

In one aspect, an IL-6a that is an antibody fragment has been chemically modified to improve or introduce a desirable property, for example PEGylation to increase half-life or incorporation.

A dAb (domain antibody) is a small monomeric antigen-binding fragment of an antibody (the variable region of an antibody heavy or light chain. VH dAbs occur naturally in camelids (e.g., camels and llamas) and can be produced by immunizing a camelid with a target antigen, isolating antigen-specific B cells and directly cloning dAb genes from individual B cells.

In one aspect, an IL-6a can be incorporated as part of a bispecific antibody prepared using methods known in the art, for example, prepared chemically or from hybrid hybridomas. Such a molecule can be a bispecific antibody fragment of a type discussed above. One non-limiting example of a method for generating a bispecific antibody is BiTETM technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction. Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, are useful, in part because they can be constructed and expressed in E. coli. Diabodies (and many other polypeptides, such as antibody fragments) of appropriate binding specificities can be readily selected using phage display (WO 1994/13804) from libraries. If one arm of the diabody is to be kept constant, for example, with a specificity directed against site II of IL-6, then a library can be made where the other arm is varied and an antibody of appropriate specificity selected.

Bispecific whole antibodies may be made by alternative engineering methods as described in described in WO 1996/27011, WO 1998/50431 and WO 2006/028936.

In one aspect, an IL-6a can comprise an antigen-binding site within a non-antibody molecule, for example, by incorporating one or more CDRs, e.g. a set of CDRs, in a non-antibody protein scaffold, as discussed further below. In one aspect, the CDRs are incorporated into a non-antibody scaffold. An IL-6 site II binding site can be provided by an arrangement of CDRs on non-antibody protein scaffolds, such as fibronectin or cytochrome B, or by randomizing or mutating amino acid residues of a loop within a protein scaffold to confer binding specificity for an IL-6 site II. Scaffolds for engineering novel binding sites in proteins are known in the art. For example, protein scaffolds for antibody mimics are disclosed in WO200034784, which describes proteins (antibody mimics) that include a fibronectin type III domain having at least one randomized loop. A suitable scaffold into which to graft one or more CDRs, e.g., a set of HCDRs, can be provided by any domain member of the immunoglobulin gene superfamily. The scaffold can be a human or non-human protein. An advantage of a non-antibody protein scaffold is that it can provide an antigen-binding site in a scaffold molecule that is smaller and/or easier to manufacture than at least some antibody molecules. Small size of a binding member may confer useful physiological properties, such as an ability to enter cells, penetrate deep into tissues or reach targets within other structures, or to bind within protein cavities of the target antigen. Typical are proteins having a stable backbone and one or more variable loops, in which the amino acid sequence of the loop or loops is specifically or randomly mutated to create an antigen-binding site that binds the target antigen. Such proteins include the IgG-binding domains of protein A from S. aureus, transferrin, tetranectin, fibronectin (e.g., using the 10th fibronectin type III domain), lipocalins as well as gamma-crystalline and other Affilin™ scaffolds (Scil Proteins, Halle, Germany). Examples of other approaches include synthetic microbodies based on cyclotides—small proteins having intra-molecular disulfide bonds, microproteins (e.g., Versabodies™, Amunix Inc., Mountain View, CA) and ankyrin repeat proteins (DARPins, e.g., from Molecular Partners AG, Zurich-Schlieren, Switzerland). Such proteins also include small, engineered protein domains such as, for example, immuno-domains (see for example, U.S. Patent Publication Nos. 2003/082630 and 2003/157561). Immuno-domains contain at least one complementarity determining region (CDR) of an antibody.

In one aspect, the antibodies disclosed herein can be modified to reduce their ability to fix complement and participate in complement-dependent cytotoxicity (CDC). In one aspect, the antibodies are modified to reduce their ability to activate effector cells and participate in antibody-dependent cytotoxicity (ADCC). In one aspect, an antibody as disclosed herein can be modified both to reduce its ability to activate effector cells and participate in antibody-dependent cytotoxicity (ADCC) and to reduce its ability to fix complement and participate in complement-dependent cytotoxicity (CDC).

### Formulation

An IL-6a, e.g., an IL-6 antibody, can be formulated in a concentration of from 0.1 mg/ml to 100 mg/ml, 0.1-80 mg/ml, 0.1 to 50 mg/ml, 0.1 mg/ml to 20 mg/ml, 0.1 mg/ml to 5 mg/ml, 0.1 mg/ml to 1 mg/ml, 1 mg/ml to 100 mg/ml; 5 mg/ml to 100 mg/ml; 5 mg/ml to 30 mg/ml; 10 mg/ml to 100 mg/ml; 10 mg/ml to 30 mg/ml; 20 mg/ml to 100 mg/ml; 30 mg/ml to 100 mg/ml; 40 mg/ml to 100 mg/ml; 50 mg/ml to 100 mg/ml; 60 mg/ml to 100 mg/ml; 1 mg/ml to 80 mg/ml; 5 mg/ml to 80 mg/ml; 10 mg/ml to 80 mg/ml; 20 mg/ml to 80 mg/ml; 40 mg/ml to 80 mg/ml; 50 mg/ml to 80 mg/ml; 60 mg/ml to 80 mg/ml; 1 mg/ml to 60 mg/ml; 5 mg/ml to 60 mg/ml; 10 mg/ml to 60 mg/ml; 20 mg/ml to 60 mg/ml; 30 mg/ml to 60 mg/ml; 40 mg/ml to 60 mg/ml; or 50 mg/l to 60 mg/ml. For example, the formulation contains about 1 mg/ml, 2 mg/ml, 5 mg/ml, 10 mg/ml, 20 mg/ml, 25 mg/ml, 30 mg/ml, 40 mg/ml, 50 mg/ml, or 55 mg/ml.

An IL-6a formulation can include other pharmaceutically acceptable excipients. In one aspect, an IL-6a, e.g., IL-6 antibody, is formulated with one or more, or all of the following: a buffer, a surfactant, and a tonicity agent (e.g., a sugar and/or a salt). In one aspect, the formulation further comprises one or more of a chelating agent, one or more of a preserving agent, one or more of an antioxidant, and/or one or more of an amino acid. In one aspect, the formulation further comprises a one or more additional therapeutic agents, e.g., a second therapeutic agent.

### Pharmaceutical Compositions and Formulations

Pharmaceutical compositions and formulations described herein can be formulated in a variety of forms. These include, for example, liquid, semi-solid, and solid dosage forms, such as liquid solutions (e.g., injectable and infusible solutions), dispersions or suspensions, including nanoparticles and liposomes. The form will generally depend on the intended mode of administration and therapeutic application. Pharmaceutical compositions described herein are typically in the form of injectable or infusible solutions, or are formulated for topical delivery, e.g., topical ocular delivery.

In one aspect, a pharmaceutical composition described herein is sterile and stable under the conditions of manufacture and storage. A pharmaceutical composition can also be tested to ensure it meets regulatory and industry standards for administration. The composition can be formulated as a solution, microemulsion, dispersion, liposome, or other ordered structure suitable to high drug (e.g., a biologic) concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients. In the case of sterile powders for the preparation of sterile injectable solutions, exemplary methods of preparation include vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be engineered by inclusion of an agent that delays absorption, for example, monostearate salts and gelatin. Such an agent may be particularly useful in a low-dose formulation. In one aspect, the formulation comprises ≤1 mg/ml of a therapeutic protein (e.g., an IL-6a, e.g., an IL-6 antibody or antigen binding fragment thereof described herein) and gelatin is included in the formulation.

In one aspect, a pharmaceutical composition or formulation is prepared with a carrier. For example, the formulation can be delivered as a controlled release formulation, delivered by an implant or a microencapsulated delivery system. Biodegradable, biocompatible polymers can be used, such as ethylene vinyl acetate, polyanhydrides, polyglycolic acid, collagen, polyorthoesters, and polylactic acid. See e.g., Sustained and Controlled Release Drug Delivery Systems, J. R. Robinson, ed., Marcel Dekker, Inc., New York, 1978.

Ophthalmic packs may be used to give prolonged contact of an ophthalmic formulation with the eye. A cotton pledget is saturated with the formulation and then inserted into the superior or inferior fornix. The formulation may also be administered by way of iontophoresis. This procedure keeps the solution in contact with the cornea in an eyecup bearing an electrode. Diffusion of the drug is effected by difference of electrical potential. Iontophoretic systems which have been used include Ocuphor®1 (Iomed Inc., USA); Eyegate® II Delivery System1 (EyeGate Pharma, USA); and Visulex®1 (Aciont Inc., USA). See Amo and Urtti, Drug Discovery Today, 13:143 (2008).

Another strategy for sustained ocular delivery is the use of gelifying agents. These materials can be delivered in a liquid form, as an eye drop or intraocular injection. After instillation the polymer undergoes a phase change and forms a semi-solid or solid matrix that releases the drug over prolonged period. The phase transition can be induced by changes in the temperature, ion concentration, or pH.

For topical ocular use, the gel forming solutions, such as Timoptic®-XE1 (Merck and Co. Inc., USA), which contains Gelrite® (purified anionic heteropolysaccharide from gellan gum); Pilogel®1 (Alcon, Inc., Switzerland) eye drops contain poly (acrylic acid); and Azasite®1 (Insite Vision, USA) have been tested clinically. These materials enhance the drug retention relative to the conventional eye drops and lead to increased drug absorption into the eye and reduced dosing frequency. See Amo and Urtti, Drug Discovery Today, 13:135-143 (2008).

### Administration

A therapy or pharmaceutical composition described herein can be delivered by injection, e.g., intravitreal, periocular, or subconjunctival injection. The therapy can be injected underneath the conjunctiva facilitating passage through the sclera and into the eye by simple diffusion. The therapy can also be injected underneath the conjunctiva and the underlying Tenon's capsule in the more posterior portion of the eye to deliver the agent to the ciliary body, choroid, and retina. The therapy may also be administered by retrobulbar injection.

In general, a pharmaceutical composition or therapy described herein can be administered to a subject, by any suitable method, such as intravenous administration as a bolus or by continuous infusion over a period of time, by intramuscular, intramuscular, intraarterial, intrathecal, intracapsular, intraorbital, intracardiac, intradermal, intraperitoneal, intrasynovial, transtracheal, subcutaneous, subcuticular, intraarticular, subcapsular, subarachnoid, intraspinal, epidural injection, intrasternal injection and infusion. Other suitable modes of administration include topical (e.g., dermal or mucosal) or inhalation (e.g., intranasal or intrapulmonary) routes. For certain applications, the route of administration is one of: intravenous injection or infusion, subcutaneous injection, or intramuscular injection. For administration to the eye, the mode of administration is topical administration to the eye, e.g., in the form of drops. Examples of devices that may contain the formulation and/or be used for adminstration of the formulation include simple eye droppers, squeeze bottles with or without metering function, and blow/fill/seal (BFS) devices such as those manufactured by Catalent (Somerset, NJ), multi-use devices using, for example tip-seal technology, silver/oligodynamic technology, sterile filters, collapsing primary containers, and the like.

An additional consideration for a container is that it provide an acceptable shelf-life once it is filled, e.g., there is an acceptably low level of evaporation and/or the formulation meets release assay specifications, e.g., specifications as described herein. In one aspect, the container is suitable to provide a shelf-life of at least two years, e.g., at least 3 years, at least 4 years, or at least 5 years, e.g., at 5° C. In one aspect, the container is suitable to provide a shelf-life of at least 3 years at 5° C. In one aspect, the container is suitable to provide a shelf-life of at least 2 months, 3 months, 4 months, 5 months, 6 months, 8 months, 10 months, or 12 months at RT. In one aspect, the the container is suitable to provide a shelf-life of at least 5 months at RT. Various suitable container materials are known in the art, for example certain plastics, for example, low density polyethylene (LDPE), high density polyethylene (HDPE), or polypropylene.

The pharmaceutical composition or therapy can be prepared for single use application in a container or can be prepared for use in a multiuse container.

A pharmaceutical composition or therapy described herein can be delivered intravitreally, e.g., to treat disorders that are associated with, for example, the posterior segment of the eye. Methods of intravitreal administration are known in the art and include, for example, intraocular injection, implantable devices.

In one aspect, the pharmaceutical composition or therapy is administered intravitreally using an implantable device. In one aspect, the pharmaceutical composition comprises a thermal stabilizer, e.g., sorbitol. In one aspect, the sorbitol is present at a concentration of ≥5% w/v.

Implantable devices can be, for example, nonbiodegradable devices such as polyvinyl alcohol-ethylene vinyl acetate polymers and polysulfone capillary fibers, biodegradable devices such as polylactic acid, polyglycolic acid, and polylactic-co-glycolic acid, polycaprolactones, and polyanhydrides. Devices can be delivered in forms such as nanoparticles, liposomes, or microspheres.

### Dosing

A pharmaceutical composition or therapy described herein can be administered as a fixed dose, as weight determined dose (e.g., mg/kg), or as an age determined dose. The pharmaceutical composition or therapy described herein can be administered, for example, four times a day; three times a day; twice a day; once every day; every other day; every third, fourth or fifth day; every week; every two weeks; every three weeks; every four weeks; every five weeks; monthly; every two months; every three months; every four months; every six months; or as needed (ad libitum).

A therapy or pharmaceutical composition can include a “therapeutically effective amount” of an agent described herein. A therapeutically effective amount of an agent can vary according to factors such as the disease state, age, sex, and weight of the individual, and the ability of the compound to elicit a desired response in the individual, e.g., amelioration of at least one disorder parameter (e.g., sign), or amelioration of at least one symptom of the disorder (and optionally the effect of any additional agents being administered). A therapeutically effective amount is also one in which any toxic or detrimental effects of the composition are outweighed by the therapeutically beneficial effects. In one aspect, a “therapeutically effective amount” is determined in a population of individuals and the amount is effective in ameliorating at least one symptom or indication of a cytokine-related disorder, e.g., an IL-6-related disorder in at least 5%, 10%, 25%, 50%, 70%, 75%, 80%, 85%, 90%, 95%, or 100% of an affected population. A pharmaceutical composition or therapy is typically administered in a therapeutically effective amount.

Pharmaceutical compositions can be administered using medical devices as known in the art, e.g., implants, infusion pumps, hypodermic needles, and needleless hypodermic injection devices. A device can include, e.g., one or more housings for storing pharmaceutical compositions, and can be configured to deliver unit doses of the IL-6a, e.g., IL-6 antibody or fragment thereof described herein, and optionally a second therapeutic agent. The doses can be fixed doses, i.e., physically discrete units suited as unitary dosages for the subjects to be treated; each unit can contain a predetermined quantity of an IL-6a, e.g., an IL-6 antibody or fragment thereof described herein, calculated to produce the desired therapeutic effect in association with a pharmaceutical carrier and optionally in association with another agent, e.g., such as those available as over the counter or prescribed products.

In one aspect, to treat a disorder described herein, the pharmaceutical composition or therapy is administered to a subject having the disorder in an amount and for a time sufficient to induce a sustained improvement in at least one sign or symptom of the disorder. An improvement is considered “sustained” if the subject exhibits the improvement over a prolonged period, e.g., on at least two occasions separated by one to four weeks. The degree of improvement can be determined based on signs or symptoms, and can also employ questionnaires that are administered to the subject, such as quality-of-life questionnaires.

Improvement can be induced by repeatedly administering a dose of the formulation until the subject manifests an improvement over baseline for selected signs and/or symptoms. In treating chronic conditions, the amount of improvement can be evaluated by repeated administration over a period of at least a month or more, e.g., for one, two, or three months or longer, or indefinitely. In treating an acute condition, the agent can be administered for a period of one to six weeks or even as a single dose.

Although the extent of the disorder after an initial or intermittent treatment can appear improved according to one or more signs or symptoms, treatment can be continued indefinitely at the same level or at a reduced dose or frequency. Treatment can also be discontinued, e.g., upon improvement or disappearance of signs or symptoms. Once treatment has been reduced or discontinued, it may be resumed if symptoms should reappear.

In one aspect, an IL-6a, preferably an IL-6 antibody, is administered intravitreally (IVT) at a dosage of 0.25 mg, 1.0 mg or 2.5 mg every 4 weeks (Q4W) for treatment of uveitis or UME, more specifically UME. In another aspect, an IL-6 antibody is administered intravitreally (IVT) at a dosage of 0.25 mg every 4 weeks (Q4W) for treatment of uveitis or UME, more specifically UME. In one aspect, an IL-6 antibody is administered intravitreally (IVT) at a dosage of 1.0 mg every 4 weeks (Q4W) for treatment of uveitis or UME, more specifically UME.

### Disease

“Ophthalmic diseases” that can be treated with an IL-6a of the invention include those diseases in which IL-6 expression, e.g., elevated IL-6 expression, is associated with the disease state or as a prerequisite to the disease state. Such diseases include those in which angiogenesis and inflammation driven by IL-6 contribute to disease pathology. This includes diseases in which IL-6 is elevated compared to normal levels, e.g., diseases in which IL-6 is elevated in the vitreous (such as, e.g., diabetic macular edema, diabetic retinopathy, and uveitis) or tissues of the eye. As described in WO2014/074905, incorporated herein by reference in its entirety, it has been previously shown that blocking the IL-6 pathway by administration of an IL-6 antibody in mouse and rat choroidal neovascularization models, which reproduce the pathologic processes underlying many IL-6 related diseases, e.g., DME, results in reduction of neovascularization to similar levels as an anti-VEGF positive control. These in vivo results demonstrate that local inhibition of IL-6 can be useful for treating ocular diseases associated with IL-6 expression and ocular diseases involving vascular leakage, e.g., macular edema.

Examples of IL-6 related diseases include certain eye diseases including, without limitation, dry eye (e.g., dry eye disease or dry eye syndrome), allergic conjunctivitis, uveitis, age-related macular degeneration (AMD) (wet (exudative) AMD or dry (atrophic) AMD), proliferative diabetic retinopathy (PDR), diabetic macular edema (DME), Rhegmatogenous retinal detachment (RRD), retinal vein occlusion (RVO), neuromyelitis optica (NMO), or myopic choroidal neovascularization. Other ocular disorders that can be treated include those caused by trauma such as corneal transplant, corneal abrasion, or other such physical injury to the eye. Other ocular disorders that can be treated include ocular cancers, e.g., cancers that affect the eye and the vicinity of the eye, e.g., the eye socket or the eyelids.

As used herein, the term “treat” refers to the administration of an agent described herein to a subject, e.g., a patient, in an amount, manner, and/or mode effective to improve a condition, symptom, or parameter associated with a disorder, e.g., a disorder described herein, or to prevent the onset or progression of a disorder, to either a statistically significant degree or to a degree detectable to one skilled in the art. The treatment can be to cure, heal, alleviate, relieve, alter, remedy, ameliorate, palliate, improve or affect the disorder, the symptoms of the disorder or the predisposition toward the disorder. An effective amount, manner, or mode can vary depending on the subject and may be tailored to the subject. Exemplary subjects include humans, primates, and other non-human mammals. A pharmaceutical composition or therapy described herein can also be given prophylactically to reduce the risk of the occurrence of a disorder or symptom or sign thereof.

In one aspect, the IL-6 related disease is an inflammatory disease. In one aspect, the disease is glaucoma.

In one aspect, the disease is ocular pain, e.g., pain associated with an ocular disease or disorder.

In one aspect, treatment of a subject also includes determining whether the subject has an IL-6 associated disease, and optionally, whether the subject is resistant to other non-IL-6inhibitory treatments such as steroids or anti-VEGF agents.

The pharmaceutical composition or therapy described herein can be administered to a subject having or at risk for such IL-6 related diseases.

The therapy or pharmaceutical composition described herein are particularly suited for use in ocular disorders, e.g. ocular disorders in which it is desired to administer the IL-6 antagonist, e.g., IL-6 antibody or fragment thereof described herein, directly to the eye, or locally to the area of the eye.

Subjects having a dry eye disorder can exhibit inflammation of the eye, and can experience scratchy, stingy, itchy, burning or pressured sensations, irritation, pain, and redness. Dry eye disorders can be associated with excessive eye watering and insufficient tear production. A pharmacdeutical composition or therapy described herein can be administered to such a subject to ameliorate or prevent the onset or worsening of one or more such symptoms. A pharmaceutical composition or therapy described herein can also be used to mitigate pain, e.g., ocular pain, such as pain due to neuroinflammation, in a subject.

The pharmaceutical composition or therapy described herein can be administered to a subject having an allergic reaction affecting the eye, e.g., a subject experiencing severe allergic (atopic) eye disease such as, e.g., allergic conjunctivitis. See also, e.g., Keane-Myers et al. (1999) Invest Ophthalmol Vis Sci, 40(12): 3041-6.

The pharmaceutical composition or therapy described herein can be administered to a subject who has or is at risk for diabetic retinopathy. See, e.g., Demircan et al. (2006) Eye 20:1366-1369 and Doganay et al. (2006) Eye, 16:163-170.

Uveitis. Uveitis includes acute and chronic forms and includes inflammation of one or more of the iris, the ciliary body, and the choroid. Chronic forms may be associated with systemic autoimmune disease, e.g., Behçet's syndrome, ankylosing spondylitis, juvenile rheumatoid arthritis, Reiter's syndrome, and inflammatory bowel disease. In anterior uveitis, inflammation is primarily in the iris (also iritis). Anterior uveitis can affect subjects who have systemic autoimmune disease, but also subjects who do not have systemic autoimmune disease. Intermediate uveitis involves inflammation of the anterior vitreous, peripheral retina, and ciliary body, often with little anterior or chorioretinal inflammation. Pan planitis results from inflammation of the pars plana between the iris and the choroid. Posterior uveitis involves the uveal tract and primarily the choroid, and is also referred to as choroiditis.

Posterior uveitis can be associated with a systemic infection or an autoimmune disease. It can persist for months and even years. The pharmaceutical composition or therapy described herein can be administered to a subject to treat any of the foregoing forms of uveitis including a complication caused by uveitis such as uvetic macular edema (UME) characterized by the accumulation of fluid in the retinal layers and/or the subretinal space. UME is the leading cause of visual impairment in cases with uveitis. See also e.g., Tsai et al. (2009) Mol Vis 15:1542-1552 and Trittibach et al. (2008) Gene Ther. 15(22): 1478-88. The IL-6 signaling is thought to regulate and amplify intraocular inflammatory and immune responses by inhibiting T-cell apoptosis and mediating differentiation of Th1 cells into Th17 cells, thought to be responsible for the pathogenesis of autoimmune disorders such as uveitis (Amadi-Obi et al., TH17 cells contribute to uveitis and scleritis and are expanded by IL-2 and inhibited by IL-27/STAT1. Nat Med 2007;13(6):711-8). Systemic inhibition of IL-6R by tocilizumab has been demonstrated to improve visual acuity and reduce macular thickness in multiple forms of uveitis, including in those patients with recalcitrant UME, and there is currently a robust supportive body of evidence, including retrospective case series and a prospective, randomized, investigator-initiated trial (Sepah et al., Primary (Month-6) outcomes of the STOP-Uveitis study: evaluating the safety, tolerability, and efficacy of tocilizumab in patients with non-infectious uveitis. Am J Ophthalmol 2017;183:71-80). Nonclinically, in a murine model of experimental autoimmune uveitis, IL-6 inhibition by IVT administration of an anti-IL-6 mAb attenuated vascular leakage and macular edema, decreased ocular production of IL-17, and improved the overall uveitis score, suggesting that local targeting of IL-6 may be sufficient for therapeutic effect (Tode J et al., Intravitreal injection of anti-Interleukin (IL)-6 antibody attenuates experimental autoimmune uveitis in mice. Cytokine 2017;96:8-15).

In one aspect, the pharmaceutical composition or therapy descriebe herein can be used to treat a subject having or at risk for age-related macular degeneration (AMD), e.g., wet (exudative) AMD or dry (atrophic) AMD. The pharmaceutical composition or therapy descriebe herein can be applied topically to the eye, injected (e.g., intravitreally) or provided systemically. See, e.g., Olson et al. (2009) Ocul Immunol Inflamm 17(3):195-200.

Diabetic macular edema (DME). Diabetic macular edema (DME) involves occlusion and leakage of retinal blood vessels, causing reduced visual acuity and potentially blindness. Standard treatments for DME include local administration of steroids or anti-VEGF antibodies. However, many patients are refractory to these therapies. The pathogenesis of diabetic macular edema involves components of angiogenesis, inflammation, and oxidative stress. IL-6 is induced by hypoxia and hyperglycemia and can increase vascular inflammation, vascular permeability, and pathologic angiogenesis. IL-6 can directly induce VEGF expression and can promote choroidal neovascularization in animal models. In DME patients, ocular IL-6 levels are positively correlated with macular thickness and disease severity. IL-6 levels are reportedly elevated in patients who fail anti-VEGF therapy while decreasing in anti-VEGF responsive patients. Accordingly, administration of an IL-6a as described herein is useful for treatment of diabetics in combination with an anti-VEGF therapeutic or as an alternative to anti-VEGF treatment, including for patients who do not respond to anti-VEGF therapy. Treatment of macular edema with an IL-6a may also improve safety by removing the need to completely inhibit either mechanism to inhibit the pathology, thus preserving some of the desired, physiological roles of each cytokine. Accordingly, local IL-6a treatment in combination with VEGF inhibition can decrease the dose frequency and reduce adverse effects of treatment.

In DME there are positive correlations between vitreal IL-6 levels and both disease severity and VEGF refractory subjects. Accordingly, an IL-6a as described herein can be used to treat DME subjects who are refractive to steroid therapy, anti-VEGF therapy, or both. Subjects that are refractive to a given therapy, e.g., steroid therapy or anti-VEGF theray, or both, do not exhibit an improvement, reduction, or amelioration of a selected symptom. In one aspect, an IL-6a, e.g., an IL-6 antibody or fragment thereof as described herein, is used in combination with anti-VEGF therapy or steroid therapy, e.g., to treat DME. Accordingly, in one aspect, the pharmaceutical composition or therapy descriebe herein can comprise an anti-VEGF agent or a steroid.

A pharmaceutical composition or therapy descriebe herein can be administered by any mode to treat an ocular disease. The pharmaceutical composition or therapy descriebe herein can be delivered by a parenteral mode. Alternatively or in addition, the pharmaceutical composition or therapy descriebe herein can be delivered directly to the eye or in the vicinity of the eye. For example, the pharmaceutical composition or therapy descriebe herein can be administered topically, intraocularly, intravitreally, e.g., by intravitreal injection, or subconjuntivally.

### Assays

Aqueous humor sample can be taken, e.g. by anterior chamber paracentesis using a fine needle and syringe or pipet (Van der Lelij A et al., Diagnostic anterior chamber paracentesis in uveitis: a safe procedure? Br J Ophthalmol 1997;81(11):976-9; Trivedi D et al., Safety profile of anterior chamber paracentesis performed at the slit lamp. Clin Exp Ophthal 2011;39(8):725-8; Kitazawa K et al., Safety of anterior chamber paracentesis using a 30-gauge needle integrated with a specially designed disposable pipette. Br J Ophthalmol 2017;101(5):548-50).

In some embodiments, the biomarker is detected in the sample using a method selected from the group consisting of FACS, Western blot, ELISA, immunoprecipitation, immunohistochemistry, immunofluorescence, radioimmunoassay, immunodetection methods, mass spectrometery, qPCR, RT-qPCR, multiplex qPCR or RT-qPCR, RNA-seq, microarray analysis, nanostring, SAGE, MassARRAY technique, and FISH, and combinations thereof. In some embodiments, the biomarker is detected in the sample by protein expression. In some embodiments, protein expression is determined by immunohistochemistry (IHC).

In one aspect, the biomarker is detected in the sample by mRNA expression. In one aspect, the mRNA expression is determined using qPCR, rtPCR, RNA-seq, multiplex qPCR or RT-qPCR, microarray analysis, nanostring, SAGE, MassARRAY technique, or FISH.

In one aspect, the sample is an AH sample.

In one aspect, the sample is obtained prior to treatment with an IL-6 antagonist. In one aspect, the AH sample is fresh or freeze-thawed.

Presence and/or level/amount/concentration of various biomarkers in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In one aspect, the sample is a clinical sample. In one aspect, the sample is used in a diagnostic assay.

In one aspect, a “reference sample” or “control sample” is a single sample or combined multiple samples from the same subject or individual that are obtained at one or more different time points than when the test sample is obtained. In one aspect, a reference sample or control sample is a combined multiple samples from one or more healthy individuals who are not the subject or individual. In one aspect, a reference sample or control sample is a combined multiple samples from one or more individuals with a disease or disorder (e.g., DME) who are not the subject or individual.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., DME) characterized by certain, molecular, pathological, histological, and/or clinical features. In one aspect, a predictive biomarker may serve as an indicator of a better or worse response to a particular treatment.

The “amount”, “concentration” or “level” of a biomarker can be measured by methods known to one skilled in the art. The level, concentration or amount of a biomarker assessed can be used to predict response to the treatment.

The term “level” is used to refer to the amount or concentration of a biomarker in a biological sample.

The term “reference level” can refer to a predetermined value. In one aspect, the reference level can be obtained by measuring the amount or concentration of a biomarker (e.g., AH IL-6) in a control sample. In one aspect, the level of AH IL-6 in a sample from a patient is increased or elevated as compared to the reference level, which indicates that the patient is likely to respond to a therapy with an IL-6 antagonist. In one aspect, the level of AH IL-6 in a sample from a patient is decreased or reduced as compared to the reference level, which indicates that the patient is not likely to respond to a therapy with an IL-6 antagonist.

Both “level” and “reference level” can be expressed in conscentrations of IL-6 in aqueous humor.

In one aspect, the term “increase”, “increased”, “elevated” or “above” refers to a level above the reference level. In one aspect, increased level refers to an overall increase of about any of 10%, 20%, 30%, 40%, 50%, 60%, 70%, 80%, 90%, 95%, 96%, 97%, 98%, 99% or greater, in the amount or concentration of a biomarker as compared to a reference level. In one aspect, the increased level refers to the increase in the amount or concentration of a biomarker in the sample wherein the increase is at least about any of 1.5X, 1.75X, 2X, 3X, 4X, 5X, 6X, 7X, 8X, 9X, 10X, 25X, 50X, 75X, or 100X the reference level. In one aspect, the increased level refers to an overall increase of greater than about 1.5 fold, about 1.75 fold, about 2 fold, about 2.25 fold, about 2.5 fold, about 2.75 fold, about 3.0 fold, or about 3.25 fold as compared to a reference level.

The term “diagnosis” is used herein to refer to the identification or classification of a molecular or pathological state, disease or condition (e.g., DME). For example, “diagnosis” may refer to identification of a particular type of ophthalmic disease. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

“Response” or whether a patient is “likely to respond” can be assessed using any endpoint indicating a benefit to the individual, including, without limitation, clinically relevant improvements of Best Corrected Visual Acuity (BCVA), reduction of central subfield thickness (CST) or central foveal thickness (CFT), reduction of fluid accumulation (e.g., retinal fluid) and/or decreased severity of diabetic retinopathy. BCVA change from baseline is a clinical endpoint widely accepted by health authorities. In clinical practice, anatomical biomarkers of retinal thickness (e.g. CST or CFT) or presence of fluids are more commonly used as efficacy biomarker for treatment decision. Retinal thickness can be measured by OCT imaging such as spectral domain optical coherence tomography (SD-OCT). Retinal fluid can be defined as itraretinal or subretinal (IRF and SRF, respectively) based on the location: in the neurosensory retinal layer for IRF and between the neurosensory retina and the underlying retinal pigment epithelium for SRF.

### Co-Therapy

An IL-6 antagonist can be used either alone or in combination with other agents in a therapy. For instance, an IL-6 antagonist may be co-administered with at least one additional therapeutic agent. In one aspect, an additional therapeutic agent is a VEGF antagonitst. In one aspect, a VEGF antagonist is an anti-VEGF antibody, e.g. bevacizumab, ranibizumab or brolucizumab. In one aspect, a VEGF antagonist is a bispecific antibody such as faricimab. In one aspect, a VEGF antagonist is a soluble VEGF receptor such as aflibercept.

Such combination therapies noted above encompass combined administration (where two or more therapeutic agents are included in the same or separate formulations), and separate administration, in which case, administration of the IL-6 antagonist can occur prior to, simultaneously, and/or following administration of the additional therapeutic agent.

### Diagnostic Kits, Assays and Articles of Manufacture

Dislosed herein are also a diagnostic kit comprising one or more reagent for determining the presence of a biomarker in a sample from a patient with an ophthalmic disease.

Disclosed herein are also an assay for identifying a patient with an ophthalmic disease to receive an IL-6 antagonist, the method comprising determining a level of IL-6 in aqueous humor in a sample obtained from the patient

Disclosed herein are also articles of manufacture comprising, packaged together, an IL-6 antagonist in a pharmaceutically acceptable carrier and a package insert indicating that the IL-6 antagonist is for treating a patient with an ophthalmic disease based on high IL-6 concentration in aqueous humor. Treatment methods include any of the treatment methods disclosed herein.

Further disclosed are a method for manufacturing an article of manufacture comprising combining in a package a pharmaceutical composition comprising an IL-6 antatonist and a package insert indicating that the pharmaceutical composition is for treating a patient with an ophthalmic disease based on high IL-6 concentration in aqueous humor.

The article of manufacture comprises a container and a label or package insert on or associated with the container. Suitable containers include, for example, bottles, vials, syringes, etc. The containers may be formed from a variety of materials such as glass or plastic. The container holds or contains a composition comprising the cancer medicament as the active agent and may have a sterile access port (for example the container may be an intravenous solution bag or a vial having a stopper pierceable by a hypodermic injection needle).

The article of manufacture may further comprise a second container comprising a pharmaceutically-acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

The article of manufacture of the present invention also includes information, for example in the form of a package insert, indicating that the composition is used for treating cancer based on a level of the biomarker(s) herein. The insert or label may take any form, such as paper or on electronic media such as a magnetically recorded medium (e.g., floppy disk) or a CD-ROM. The label or insert may also include other information concerning the pharmaceutical compositions and dosage forms in the kit or article of manufacture.

It is understood that the singular form “a”, “an”, and “the” includes plural references unless indicated otherwise.

The present invention is further described by reference to the following non-limited figures and examples.

### EXAMPLES

### Example 1: DOVETAIL (BP40899) Clinical Trial

DOVETAIL (BP40899) is the first study where RO7200220 is administered to humans. This Phase I, multi-center, non-randomized, open-label multiple ascending dose study of RO7200220 with unilateral IVT (intravitreal) administration is designed to evaluate a range of IVT doses expected to be safe and potentially effective in patients with DME and UME. The overall study will provide data for safety and tolerability, as well as characterization of the pharmacokinetics (PK), systemic anti-drug antibodies (ADA), duration of target engagement (PD) in aqueous humor, and early signals of biological activity. This study is being conducted in adult male and female participants with center-involving DME (Parts 1, 2 and 3) or UME (Part 4). The therapeutic benefit for participants enrolled in this first-in-human study is unknown. DOVETAIL's primary objective is the assessment of the safety and tolerability of RO7200220 as monotherapy (DME and UME population) and in combination with ranibizumab (DME population only). The secondary objective is to investigate the systemic PK of RO7200220. Efficacy variables such as BCVA and anatomical outcomes in the eye (e.g. Central subfield thickness by OCT) are analyzed as exploratory objectives. Aqueous humor samples are collected by anterior chamber paracentesis in all patients participating in DOVETAIL. Analysis of concentration of IL-6 in aqueous humor of study participants is performed as an exploratory objective.

In Part 1, RO7200220 was administered as monotherapy by IVT injection twice, 6 weeks apart in the DME study eye eligible to enroll. Six provisional escalating dose levels were administered sequentially to six different cohorts. Each participant within a given cohort received RO7200220 at the assigned dose level at a constant volume of 50 μL in the specified single study eye:
Cohort 1:0.01 mg RO7200220 (starting dose)
Cohort 2:0.05 mg RO7200220
Cohort 3:0.25 mg RO7200220
Cohort 4:1 mg RO7200220
Cohort 5:2.5 mg RO7200220
Cohort 6:5 mg RO7200220.

Each cohort enrolled a minimum of 3 participants (i.e., minimum required for a decision to escalate to the next dose level) and up to a maximum of 6 participants.

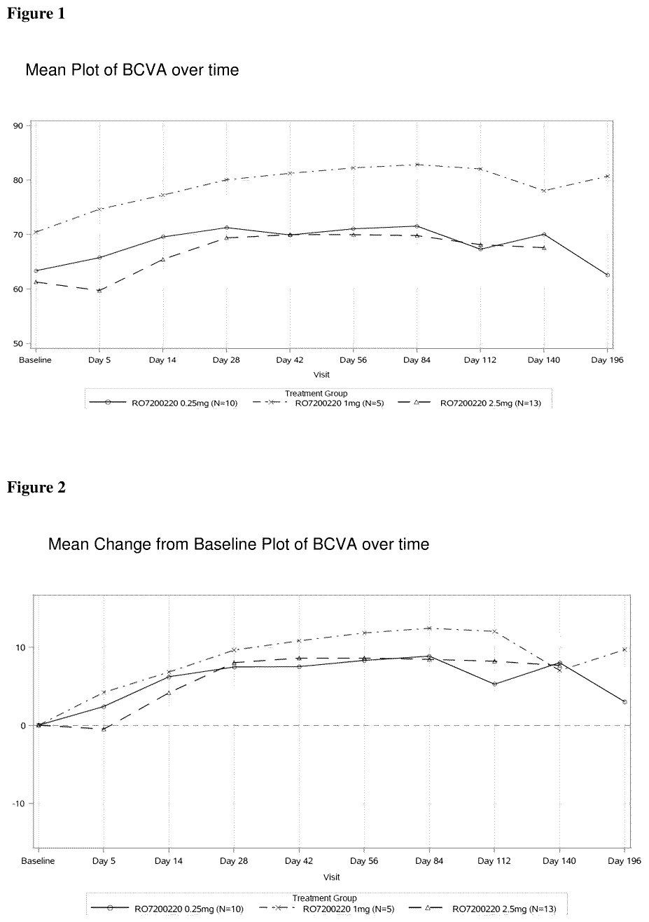

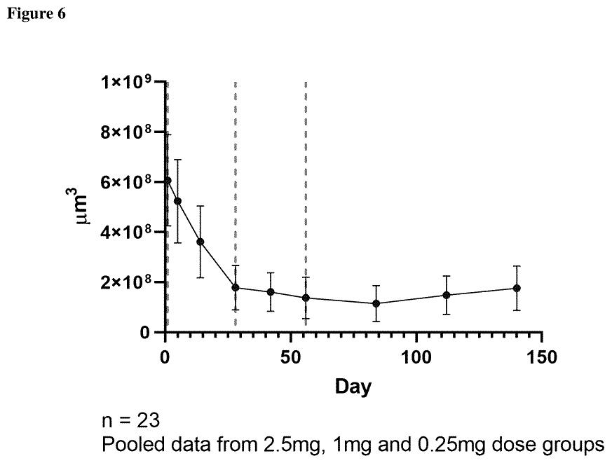

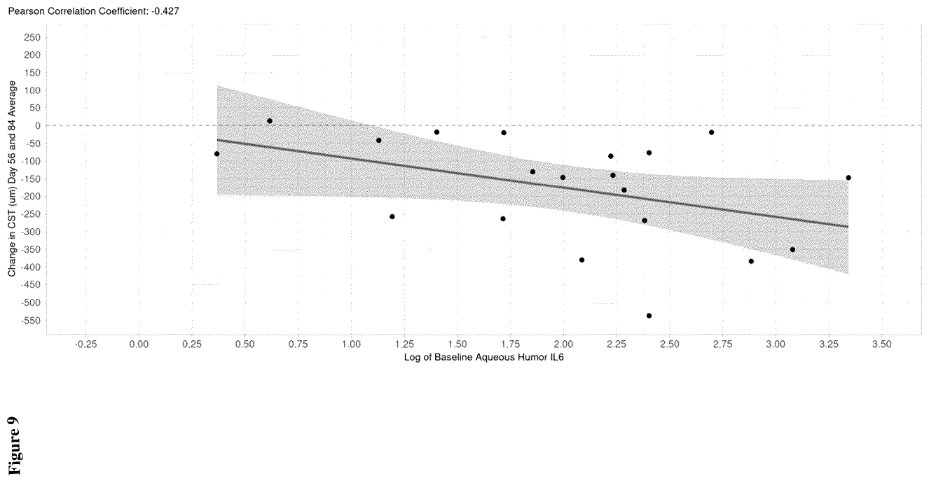

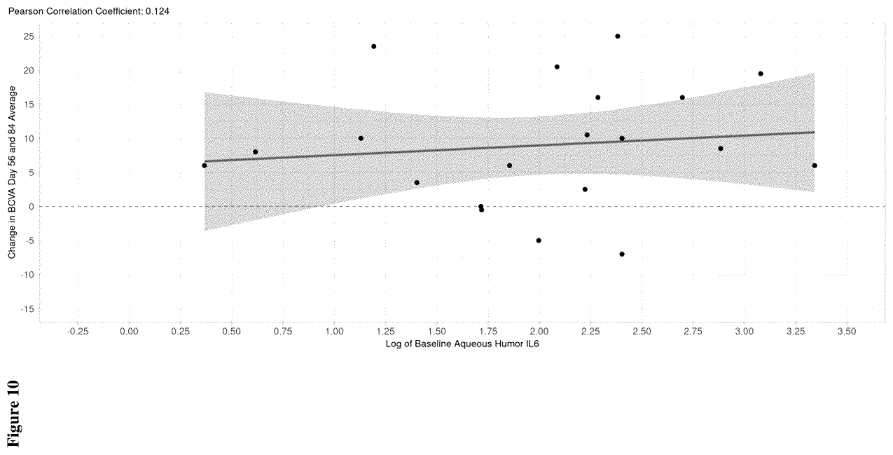

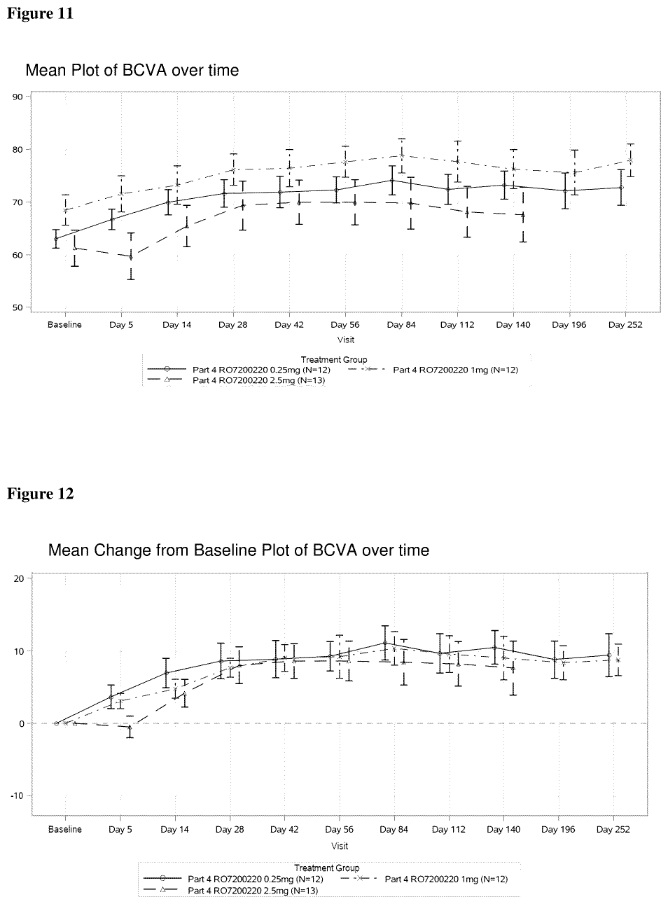

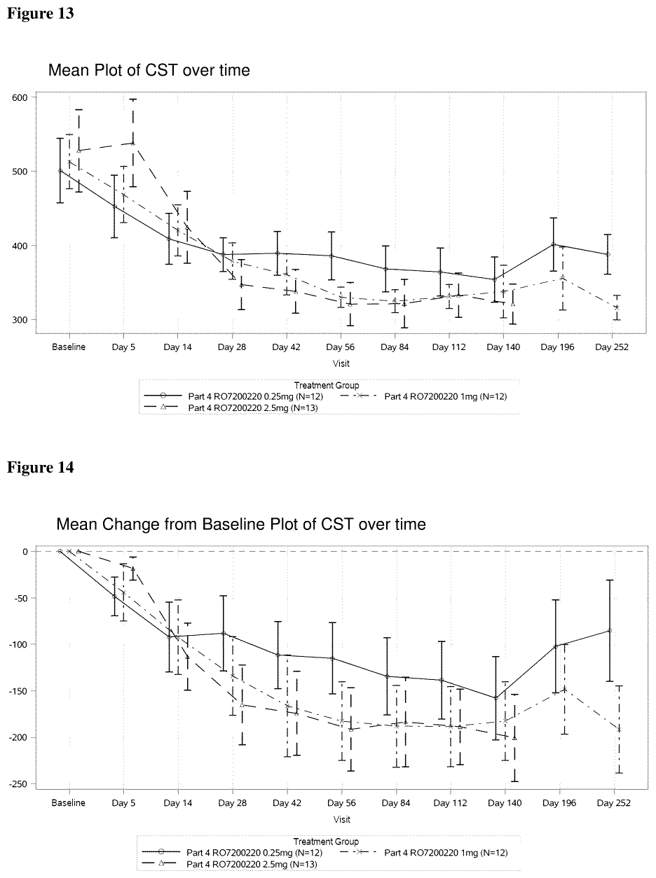

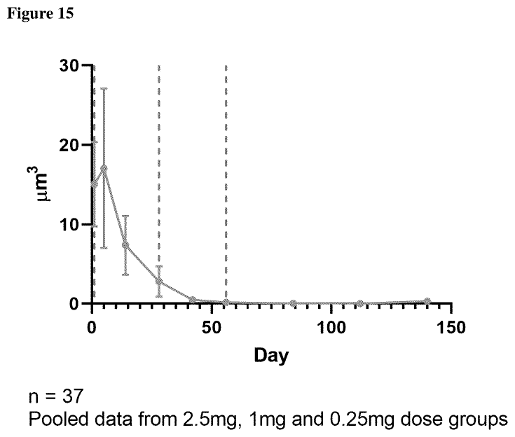

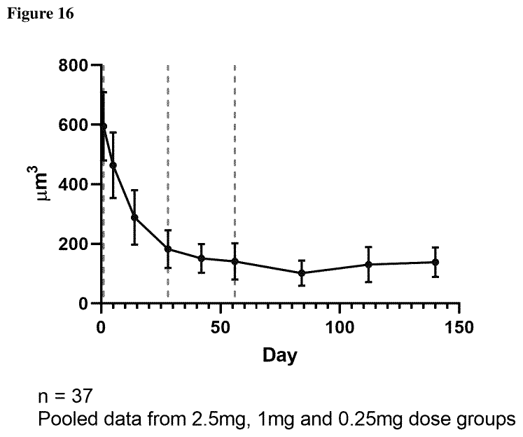

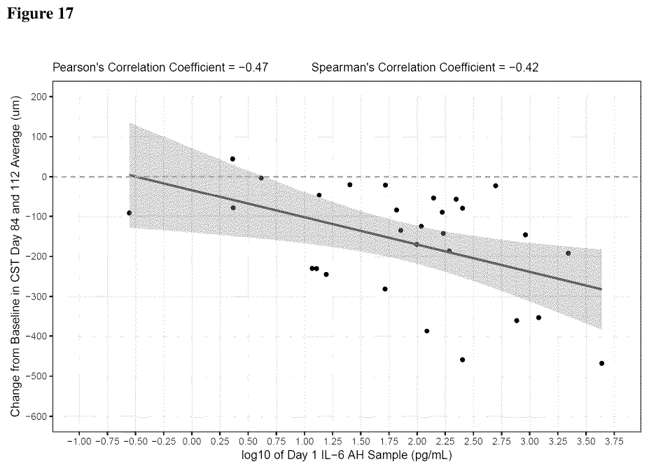

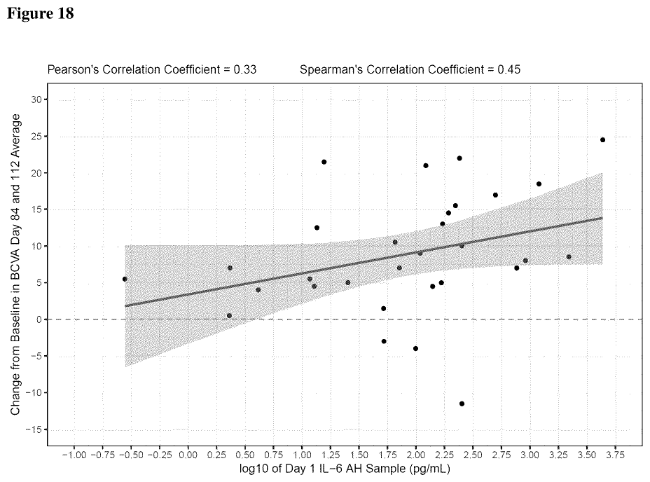

Part 4 evaluated RO7200220 as monotherapy in the UME population. Three different doses were investigated in participants with UME (0.25 mg, 1 mg, and 2.5 mg administered IVT 3 times, 4 weeks apart). A total of 37 participants with UME received doses of RO7200220between 0.25 and 2.5 mg every 4 weeks (Q4W). Data from 28 participants were evaluated at the first cutoff point (FIGS. 1-6 and FIGS. 9-10); data from all 37 parcitipants were evaluated at the second cutoff point (FIGS. 11-18). Clinically meaningful improvements in BCVA and CST were observed with an acceptable safety profile in Phase I DOVETAIL participants at 0.25, 1, and 2.5 mg dose levels. The dose 2.5 mg was identified as the maximum tolerated dose.

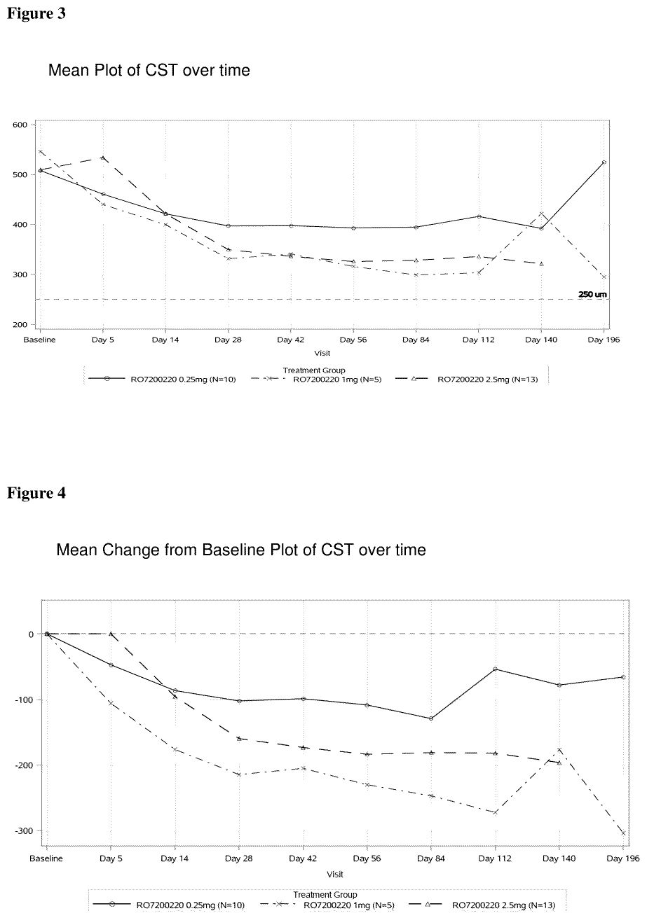

Signs of clinical efficacy were consistently observed across cohorts in Part 4 of the DOVETAIL study. Encouraging numerical improvements in BCVA (increase, FIGS. 1, 2, 11 and 12) and CST (decrease, FIGS. 3, 4, 13 and 14) were detected in the overall UME population (Part 4), without an obvious return to baseline at the end of the study. In particular, the mean change from baseline in BCVA was of 9.9 [9.4] letters (standard deviation 8.9 [9.3]) at Day 84 (4 weeks after the third and last study treatment administration, n=36 [23]). The mean change from baseline in CST was of −169.3 μm [−170.5 μm] (standard deviation 147.5 [143.8]) at Day 84 (4 weeks after the third and last study treatment administration, n=34 [21]). The numbers in square brackets are those at the first cutoff point.

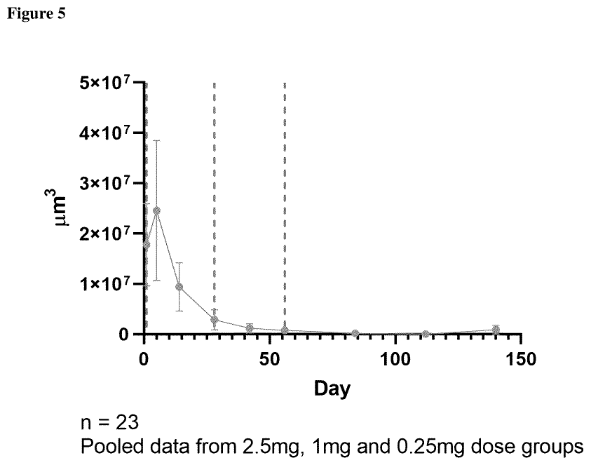

In addition, analysis of SD-OCT images showed resolution of both subretinal and intraretinal fluids (SRF and IRF, FIGS. 5, 6, 15 and 16) in the UME patients treated with RO7200220, further supporting the beneficial clinical effect of inhibiting IL-6 in treatment of UME.

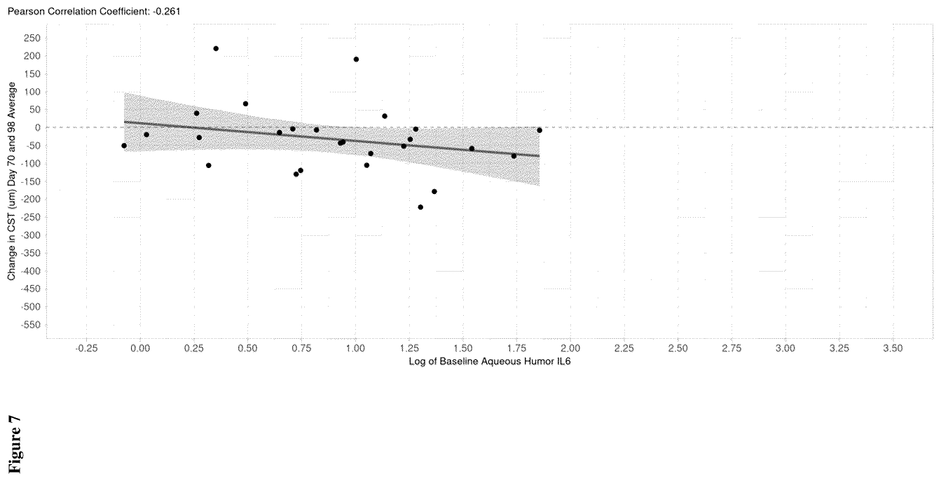

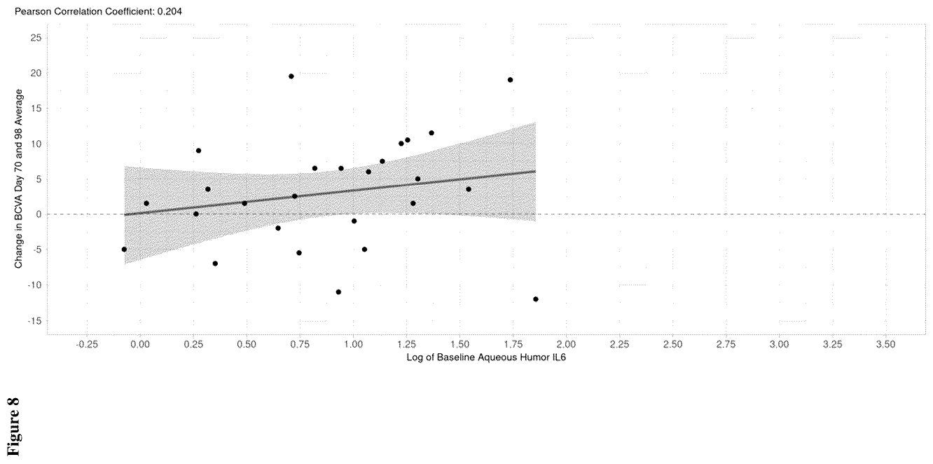

A preliminary analysis of the available data explored whether baseline levels of IL-6 in aqueous humor is associated with central subfield thickness (CST) (FIGS. 7, 9 and 17), a commonly used variable to assess anatomical response in retinal diseases such as DME and UME patients (Schmidt-Erfurth U et al.: Guidelines for the Management of Diabetic Macular Edema by the European Society of Retina Specialists (EURETINA). Ophthalmologica 2017;237:185-222), and with Best Corrected Visual Acuity (BCVA) (FIGS. 8, 10 and 18), the typical primary clinical efficacy endpoint for regulatory approval of drugs to treat this disease. One can observe an association between the AH IL-6 concentration (log scale) at baseline and the decrease of CST for both diseases, DME and UME. Furthermore, one can also observe an association between the AH IL-6 concentration (log scale) at baseline and the increase in BCVA for DME and UME. The direction of the association of AH IL-6 and CST and BCVA is in concordance with a likely higher efficacy of inhibiting IL-6 in patients with high AH IL-6.

1. A method for treating a patient with an ophthalmic disease, wherein the patient had been determined to have an increased level of IL-6 in aqueous humor (AH IL-6) in a sample obtained from the patient relative to a reference level, the method comprising administering to the patient an amount of an interleukin-6 (IL-6) antagonist.

2. The method of claim 1, wherein the IL-6 antagonist is formulated as a pharmaceutical composition suitable for administering in the eye of the patient.

3. The method of claim 2, wherein the pharmaceutical composition is suitable for administering intravitreally, intraocularly, or subconjunctivally.

4. The method of claim 1, wherein IL-6 level is determined in an aqueous humor sample collected by anterior chamber paracentesis.

5. The method of claim 1, wherein the ophthalmic disease is diabetic macular edema (DME), diabetic retinopathy, dry eye, allergic conjunctivitis, uveitis, uveitic macular edema (UME), age-related macular degeneration (AMD), proliferative diabetic retinopathy (PDR), Rhegmatogenous retinal detachment (RRD), retinal vein occlusion (RVO), neuromyelitis optica (NMO), myopic choroidal neovascularization, an ocular cancer, corneal transplant, corneal abrasion, or physical injury to the eye.

6. The method of claim 5, wherein the ophthalmic disease is diabetic macular edema (DME).

7. The method of claim 1, wherein the IL-6 antagonist is an anti-IL-6 or anti-IL-6 receptor (IL-6R) antibody or antigen binding fragment thereof.

8. The method of claim 7, wherein the IL-6 antagonist is an anti-IL-6 antibody or antigen binding fragment thereof.

9. The method of claim 7, wherein the anti-IL-6 antibody comprises:
i) VH CDR1 comprising the sequence of SEQ ID NO:1, VH CDR2 comprising the sequence of SEQ ID NO:2, and VH CDR3 comprising the sequence of SEQ ID NO:3; and
ii) VL CDR1 comprising the sequence of SEQ ID NO:4, VL CDR2 comprising the sequence of SEQ ID NO:5, and VL CDR3 comprising the sequence of SEQ ID NO:6.

10. The method of claim 9, wherein the anti-IL-6 antibody comprises a heavy chain variable region comprising the sequence of SEQ ID NO: 7 and a light chain variable region comprising the sequence of SEQ ID NO:8.

11. The method of claim 10, wherein the IL-6 antibody comprises a heavy chain comprising the sequence of SEQ ID NO:9 and a light chain comprising the sequence of SEQ ID NO:10.

12. The method of claim 1, wherein the sample is a sample obtained from the patient prior to the treatment with an IL-6 antagonist.

13. The method of claim 1, further comprising administration of an effective amount of a second therapeutic agent.

14. The method of claim 13, wherein the second therapeutic agent is a VEGF antagonist.

15. The method of claim 14, wherein the VEGF antagonist is an anti-VEGF antibody.

16. A method for treating a patient with uveitis or uveitic macular edema, the method comprising administering to the patient an amount of IL-6 antagonist.

17. The method of claim 16, wherein the IL-6 antagonist is formulated as a pharmaceutical composition suitable for administering in the eye of the patient.

18. The method of claim 17, wherein the pharmaceutical composition is suitable for administering intravitreally, intraocularly, or subconjunctivally.

19. The method of claim 16, wherein the IL-6 antagonist is an anti-IL-6 or anti-IL-6 receptor (IL-6R) antibody or antigen binding fragment thereof.

20. The method of claim 19, wherein the IL-6 antagonist is an anti-IL-6 antibody or antigen binding fragment thereof.

21. The method of claim 19, wherein the anti-IL-6 antibody comprises:
i) VH CDR1 comprising the sequence of SEQ ID NO:1, VH CDR2 comprising the sequence of SEQ ID NO:2, and VH CDR3 comprising the sequence of SEQ ID NO:3; and
ii) VL CDR1 comprising the sequence of SEQ ID NO:4, VL CDR2 comprising the sequence of SEQ ID NO:5, and VL CDR3 comprising the sequence of SEQ ID NO:6.

22. The method of claim 21, wherein the anti-IL-6 antibody comprises a heavy chain variable region comprising the sequence of SEQ ID NO:7 and a light chain variable region comprising the sequence of SEQ ID NO:8.

23. The method of claim 22, wherein the IL-6 antibody comprises a heavy chain comprising the sequence of SEQ ID NO:9 and a light chain comprising the sequence of SEQ ID NO:10.

24. The method of claim 16, wherein the IL-6 antagonist is administered intravitreally (IVT) at a dosage of 0.25 mg, 1.0 mg or 2.5 mg every 4 weeks (Q4W).


SEQUENCE LISTING (10 sequences)

SEQ ID NO: 1 - protein, 10 aa, synthetic construct
GYVLPNYLIE

SEQ ID NO: 2 - protein, 10 aa, synthetic construct
VTTPGGGTIN

SEQ ID NO: 3 - protein, 13 aa, synthetic construct
SRWDPLYYYALEY

SEQ ID NO: 4 - protein, 15 aa, synthetic construct
RASESVDNYGIPFMN

SEQ ID NO: 5 - protein, 7 aa, synthetic construct
AASNRGS

SEQ ID NO: 6 - protein, 9 aa, synthetic construct
QQSEEVPLT

SEQ ID NO: 7 - protein, 122 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSS

SEQ ID NO: 8 - protein, 111 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIK

SEQ ID NO: 9 - protein, 448 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVAQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 218 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC